(19) United States
(12) Patent Application Publication
(10) Pub. No.: US 2020/0016200 A1
(43) Pub. Date: Jan. 16, 2020

(54) MODULATING CYTOTOXIC CELL LYTIC GRANULE POSITIONING TO PROMOTE DIFFUSE KILLING IN CELLULAR THERAPIES

(71) Applicant: Baylor College of Medicine, Houston, TX (US)

(72) Inventors: Hsiang Ting Hsu, Houston, TX (US); Jordan Scott Orange, Houston, TX (US); Emily Margaret Mace, Houston, TX (US); Ashley Mentlik James, Houston, TX (US)

(21) Appl. No.: 16/335,565

(22) PCT Filed: Oct. 3, 2017
(86) PCT No.: PCT/US17/54986

§ 371 Date: Mar. 21, 2019

Related U.S. Application Data
(60) Provisional application No. 62/403,281, filed on Oct. 3, 2016.

(51) Int. Cl. A61K 35/17; A61K 31/517; C07K 16/28

(52) U.S. Cl. CPC A61K 35/17; C07K 2317/76; C07K 16/2845; A61K 31/517

(57) ABSTRACT

Embodiments of the disclosure concern methods and compositions for enhancing therapy for a medical condition, such as cancer. In particular embodiments, the therapy comprises cellular therapy, and the disclosure concerns manipulation of the cells to release contents of lytic granules in a diffuse manner to promote killing of nearby cells in dispersed directions. In specific cases, the disclosure concerns exposing the cells to an inhibitor of granule transport molecules, such as dynein, for example.

This application claims priority to U.S. Provisional Patent Application Ser. No. 62/403,281, filed Oct. 3, 2016, which is incorporated by reference herein in its entirety.

### STATEMENT REGARDING FEDERALLY SPONSORED RESEARCH OR DEVELOPMENT

This invention was made with government support under AI067946 awarded by National Institutes of Health. The government has certain rights in the invention.

### TECHNICAL FIELD

Embodiments of the disclosure concern at least the fields of cell biology, molecular biology, immunology, and medicine.

### BACKGROUND

Natural killer (NK) cells are cytotoxic lymphocytes that play a critical role in the elimination of transformed and virally infected cells (Vivier et al., 2008). NK cells express numerous germline encoded activating receptors including the natural cytotoxicity receptors (NCRs), CD16 (IgG Fc receptor), and adhesion receptors such as LFA-1 integrin (Lanier, 2005). The activating receptors recognize signatures of cell stress or disease, including IgG opsonization via CD16, to promote signaling pathways, which when surpassing critical thresholds, initiate a stepwise series of cellular events that can result in secretion of specialized secretory lysosomes termed “lytic granules” (Mace et al., 2014). After adhering to a prototypical target cell, NK cells rapidly reorient their lytic granules to the microtubule-organizing center (MTOC) using dynein motors (Ham et al., 2015; James et al., 2013; Mentlik et al., 2010; Zhang et al., 2014). This is followed by polarization of the lytic granules and MTOC to the interface formed with the target cell (also known as the lytic immunological synapse) (Katz et al., 1982; Laan et al., 2012; Yi et al., 2013) and then degranulation (Liu et al., 2011), which facilitates fatal secretion of the pore-forming molecule perforin and lytic enzymes onto the target cell.

Among cells that contain lysosome-related organelles, NK cells and cytotoxic T lymphocytes (CTLs) are the only known to converge their granules before secreting the granule contents onto target cells (Mentlik et al., 2010; Ritter et al., 2015). Granule convergence in NK cells can be triggered by the adhesion molecule LFA-1 as well as by other activation receptors and precedes any commitment to cytotoxicity. The dynein-dependent minus-end directed movement of lytic granules is dependent on Src family kinase activity as well as signaling downstream of LFA-1 signaling (James et al., 2013; Zhang et al., 2014), but is independent of actin and microtubule reorganization and other signals required for cytotoxicity (James et al., 2013; Mentlik et al., 2010).

In comparison to lymphocytes, mast cells and melanocytes undergo multi-directional dispersion of secretory organelles (Marks et al., 2013), presumably allowing for efficient distribution of their granule contents. In these cells, convergence prevents (not promotes) degranulation (Nascimento et al., 2003). The early, rapid and regulated convergence of lytic granules in cytotoxic cells of both the innate (Mentlik et al., 2010) and adaptive (Ritter et al., 2015) arms of the immune system suggests it is an evolutionarily conserved mechanism. Any contribution of this mechanism to cytotoxicity, however, has not been identified or proven.

The disclosure satisfies a long-felt need in the art by providing methods and compositions for manipulating lytic granular positioning to enhance cellular therapy for medical conditions, such as cancer.

### BRIEF SUMMARY

Embodiments of the disclosure concern modulation of cytotoxic cell lytic granule convergence to promote diffuse killing in therapy, such as therapy that comprises the use of cells. Methods and compositions are encompassed herein in which lytic granules in cells comprising the granules are prevented from converging, and their positioning remains diffuse in a cell. This allows the granules to degranulate multi-directionally upon entry into a tumor and upon trigger by recognition of a tumor cell. This allows a single therapy cell to mediate substantive collateral damage within a tumor environment and kill additional tumor cells.

In one embodiment, there is a method of enhancing a cellular therapy for cancer for an individual, comprising the step of exposing cells of the cellular therapy to an effective amount of one or more agents that inhibits convergence of lytic granules in the cells, controls positioning of lytic granules in the cells, or maintains lytic granules near the surface of the cells. The cells may be immune cells (such as T cells, NK cells, NK T cells, cytotoxic innate lymphoid cells, or a mixture thereof) or cytotoxic cells. The cells may be from cell lines and/or may be allogeneic or autologous to an individual. In specific cases, the one or more agents are exposed to the cells ex vivo. The one or more agents may be expressed from a non-endogenous molecule in the cells, such as an expression vector in the cell or a molecule that has incorporated into the genome of the cell.

In specific cases, the one or more agents are one or more of the following: a) an inhibitor of a motor protein involved in transport of the granules, b) an inhibitor of an activating receptor of the motor protein; c) an inhibitor of a signaling molecule for the motor protein; and/or d) an inhibitor of a receptor that induces a signaling molecule for the motor protein function. In particular cases, the one or more agents are one or more of the following: a) an inhibitor of dynein; b) an inhibitor of an activating receptor of dynein; c) an inhibitor of a signaling molecule for dynein function; and/or d) an inhibitor of a receptor that induces a signaling molecule for dynein function. In specific aspects, the inhibitor is an inhibitor of dynein, dynactin, HkRP3, Rab7, RILP, ORP1L, Pyk2, CLP170, leupaxin, LFA1, CD11a, CD18, CD54. Src, NIK, RASGRP1, PTEN, ILK, PINCH1, γ-parvin, paxillin, RhoGEF7; CDC42, Paro, aPKC, GSK3β, APC, IQGAP1, CLIP-170, Arl8b, or a combination thereof. When the agent is an inhibitor of dynein, the dynein that is inhibited may be heavy chain, intermediate chain, light intermediate chain, or light chain. The dynein may be DYNC1H1, DYNC2H1, DYNC1I1, DYNC1I2, DYNC1LI1, DYNC1LI2, DYNC2LI1, DYNLL1, DYNLL2, DYNLRB1, DYNLRB2, DYNLT1, or DYNLT3. In specific cases, the inhibitor of dynein is a ciliobrevin.

In one embodiment, there is a method of enhancing a therapy for cancer in an individual, comprising the step of administering to the individual an effective amount of one or more agents that inhibits convergence of lytic granules in the cells, controls positioning of lytic granules in the cells, or maintains lytic granules near the surface of the cells in immune cells or cytotoxic cells of the individual. In specific cases, the therapy is an antibody, a fragment of an antibody, a soluble ligand or receptor, a cell permeable peptide, or a mixture thereof. The antibody may be an anti-LFA-1 antibody, an anti-CD18 antibody, an antibody to CD11a, or a combination thereof, as examples.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which

FIGS. 1A-1D. LFA-1 but not CD16 engagement induces lytic granule convergence in NK cells. Fixed cell confocal microscopy of YTS-CD16 (1A) and ex vivo NK (eNK) cells (1B) incubated with S2, S2-Antiserum (S2 antiserum-labeled S2 cells), S2-IC1 (S2-ICAM1) or S2-IC1-Antiserum (S2 antiserum-labeled S2-ICAM-1) cells. The NK cells appear by themselves when incubated with uncoated S2 as they did not adhere to the NK cells. Quantitative analyses of lytic granule distance from the MTOC are shown as a feature of the degree of granule convergence in YTS-CD16 (1C) and eNK (1D) cells. Data represent 30 cells per group from three independent experiments for YTS-CD16 cells and three healthy donors for eNK cells. Gray points in each condition indicate the representative conjugates shown in (1A) and (1B).

FIGS. 2A-2F. CD16 engagement induces conjugate formation and degranulation in human NK cells. For conjugation assay, numbers indicate the percentage of YTS-CD16 (2A) cells, NK92-CD16 (2B) cells and previously cryopreserved eNK (2C) cells in conjugates. Data represent results from three independent experiments using NK cell lines or eNK cells from three healthy donors. For degranulation assay, combined results from 7 and 3 experiments for YTS-CD16 (2D) and NK92-CD16 (2E) cells showed significantly higher degranulation level of NK cells co-cultured with S2-IC1-IgG cells compared to S2-IgG cells. (2F) Data from three healthy donors showed comparable degree of degranulation by eNK cells co-incubated with S2-IgG and S2-IC1-IgG cells.

FIGS. 3A-3E. Engagement of LFA-1 and CD16 induces more targeted degranulation at the IS than CD16 alone. Fixed cell imaging flow cytometry of YTS-CD16 cells conjugated with S2-IgG or S2-IC1-IgG cells (3A). Quantitative analyses of area, mean fluorescence intensity (MFI) and total fluorescence intensity (area×NMI) of LysoTracker Red (lytic granules) (3B) and CD107a (3C) staining at the immunological synapse are shown as a feature of directed degranulation of YTS-CD16 cells. Data represent pooled results from three independent experiments (N>100 cells/group). Live cell confocal microscopy of YTS-CD16 cells transduced with a degranulation indicator LAMP1-pHluorin construct conjugated with S2-IgG or S2-IC1-IgG (3D) cells, or 10 μm polystyrene beads coated with anti-CD16 or anti-CD18+anti-CD16 antibody (3E). Magenta, target cells; red, LysoTracker Red (lytic granules); green, pHluorin (degranulation events).

FIGS. 4A-4E. Targeted secretion of lytic granules promotes more killing of the target cells. Live cell confocal microscopy of YTS-CD16 cells conjugated with S2-IgG (4A) or S2-IC1-IgG (4B) cells. NK cells were mixed with the target cells immediately before the imaging process. Cell mixtures were imaged every 5 min for 2 hours. Time zero represents the time when imaging started. Yellow, S2-IgG or S2-IC1-IgG cells; red, LysoTracker Red (lytic granules); blue, SYTOX Blue viability dye. Quantitative analyses of viable cells are shown as a feature of the differential killing efficiency. Live granule tracking in YTS-CD16 cells conjugated with S2-IgG and S2-IC1-IgG cells respectively (4C). Each point indicates one independent experiment using YTS-CD16 (4D) and eNK (4E) cells (N>300 cells/group).

FIGS. 5A-5D. Non-directed degranulation outside of the IS increases bystander killing of the neighboring cells. Live cell confocal microscopy of YTS-CD16 cells incubated with S2 cells as innocent bystanders and S2-IgG (5A) or S2-IC1-IgG (5B) cells as activating targets. NK cells were mixed with the target cells immediately before the imaging process and imaged every 5 min for 2 hours. Yellow, IgG-labeled S2 or S2-IC1 cells; green, bystander S2 cells; red, LysoTracker Red (lytic granules); blue, SYTOX Blue viability dye. Quantitative analyses of percent non-specific killing of S2 cells over total lysis are shown as a feature of collateral damage to the bystander S2 cells by YTSCD16 (5C) and eNK (5D) cells. Each point indicates one independent experiment (N>400 cells/group).

FIGS. 6A-6E. CD16 ligation alone induces similar IS geometry to the IS engaging both LFA-1 and CD16. (6A) Example confocal microscopy images of YTS-CD16 cells mixed with differentially labeled S2 cells as performed in FIG. 5. Cell outlines are drawn to indicate the perimeter of the conjugate analyzed (yellow, target; red, effector). (6B-6E) Quantitative analysis of: the total fluorescence intensity of LysoTracker Red at the synapse (6B), the length of synapse (6C) and the percentage of the perimeter of the cell involved in the synapse from the standpoint of the effector (6D) or the target (6E).

FIGS. 7A-7I. Ciliobrevin D inhibits granule convergence in NK cells. Fixed cell confocal microscopy of YTS (7A), NK92 (7B) and eNK (7C) cells conjugated with their respective target cells after DMSO or ciliobrevin D treatment (100 μM). Red, anti-perforin; blue, 721.221 or K562 cells; green, anti-α-tubulin. Quantitative analyses of the average lytic granule distance from the MTOC and its standard deviation are shown as a feature of the degree of granule convergence in YTS (7D, 7G), NK92 (7E, 7H) and eNK (7F, 7I) cells. Data represent pooled results from two independent experiments for YTS cells and NK92 cells, and two healthy donors for eNK cells.

FIGS. 8A-8C. Ciliobrevin D increases bystander killing of the neighboring cells. Flow cytometry-based cytotoxicity assay of NK cells treated with ciliobrevin D or DMSO control were performed as described in Materials and methods. Raji cells were used as non-susceptible bystander cells to measure the degree of collateral damage caused by non-directional degranulation. Specific lysis of the corresponding susceptible targets 721.221 and K562 cells by YTS (8A, left) and NK92 (B, left) cells were not affected with ciliobrevin D treatment, whereas the non-specific lysis of Raji cells by YTS (8A, right) and NK92 (8B, right) cells increased after ciliobrevin D treatment compared to the DMSO control. The cytotoxic function of eNK cells against K562 cells was also not affected (8C, left) and the bystander killing of Raji cells was increased (8C, right). Data from three independent experiments for YTS and NK92 cells and three healthy donors for eNK cells were shown. Colors denote individual experiments or donors.

FIGS. 9A-9D. LFA-1 blockade increases bystander killing. Live cell confocal microscopy of antibody-dependent cellular cytotoxicity by eNK cells pre-treated with murine IgG1 mAb control (9A) or LFA-1 blocking mAb (clone TS1/22, 9B). Green, RTX-coated Raji cells (NK-inciting targets); yellow, uncoated Raji cells (bystanders); red, LysoTracker Red (lytic granules); blue, SYTOX Blue viability dye. NK cells were mixed with the target cells immediately before the imaging process and imaged every 4 min for 4 hours. Quantitative analyses of viable cells are shown to demonstrate specific (9C, left) versus non-specific (9C, right) killing by eNK cells. Data represent combined results from three healthy donors. Standard 4-hour 51Cr cytotoxicity assay of NK cells treated with LFA-1-blocking mAb or murine IgG control (9D). Each dot represents an individual healthy donor.

FIGS. 10A-10B. LFA-1 but not CD16 engagement induces lytic granule convergence in NK92 cells. Fixed cell confocal microscopy of CD16-expressing human NK cell line NK92 (NK92-CD16) cells conjugated with differentially labeled S2 cells. Red, CellTracker Orange (S2 cells); green, antiperforin; blue, anti-α-tubulin. Data represent at least 25 cells per group from one experiment (10B). Error bars show ±SD. Gray points in each condition indicate the representative cells shown in (10A).

FIGS. 11A-11C. CD16 engagement induces conjugate formation and degranulation in human NK cells. For conjugation analysis, YTS-CD16 (11A) or NK92-CD16 (11B) cells were incubated with the S2, S2-Antiserum (S2 antiserum-labeled S2 cells), S2-IC1 or S2-IC1-Antiserum (S2 antiserum-labeled S2-ICAM1) cells for 0, 10, 30, or 60 min, vortexed, fixed and analyzed by flow cytometry to determine the percentage of NK cells in conjugates. Data represent the combined results from three independent experiments. For degranulation analysis, YTS-CD16, NK92-CD16 or eNK cells were mixed with S2, S2-IgG (IgG-labeled S2 cells), S2-IC-1 or S2-IC1-IgG (IgG-labeled S2-ICAM1) cells at 37° C. for 2 hours in the presence of anti-CD107a antibody and GolgiStop (BD) and analyzed using flow cytometry. Numbers in the representative flow cytometry plots indicate the percentage of CD107a positive NK cells among all NK cells acquired (11C).

FIGS. 12A-12B. Transfer rate and labeling efficiency of anti-S2 IgG on Drosophila S2 cells. Plain S2 cells were either stained with yellow vital dye or labeled with anti-S2 IgG directly conjugated with Alexa Fluor® 568 (Thermo Fischer). The yellow-labeled S2 cells and S2-IgG 568 cells were then mixed at different concentrations at a 1:1 ratio. Cell mixtures were incubated at 37° C. and analyzed at 1- and 2-hr time points. Representative flow plots showing the gating strategy for flow cytometry analysis (12A). Cells were first gated for singlets and further analyzed for the transfer rate of anti-S2 IgG 568 from the IgG-labeled S2 to yellow-labeled S2 cells (group a). The labeling efficiency of anti-S2 IgG 568 on the plain S2 cells was also analyzed as a positive control (group b). Representative data from three independent experiments were shown (12B). The percentage of anti-S2 IgG 568 positive yellow-labeled S2 cells was shown on the left, demonstrating the transfer rate of anti-S2 IgG from IgG-labeled to IgG-unlabeled S2 cells during co-incubation. The percentage of anti-S2 IgG 568 positive S2-IgG 568 cells was shown on the right demonstrating the labeling efficiency of anti-S2 IgG antibodies.

FIGS. 13A-13E. The effect of ciliobrevin D on degranulation and the increase of bystander killing by NK cells. Flow cytometry-based degranulation assay of NK cells treated with ciliobrevin D or DMSO as vehicle control. 721.221 cells were used as targets for YTS cells and K562 cells were used as targets for NK92 and eNK cells. DMSO or ciliobrevin D-treated NK cells were mixed with their respective target cells and co-cultured at 37° C. for 2 hours in the presence of anti-CD107a antibody, GolgiStop (BD) and analyzed using flow cytometry. Numbers in the representative flow cytometry plots indicate the percentage of CD107a positive eNK (13A), YTS (13B), or NK92 (13C) cells. Standard 4-hour 51Cr cytotoxicity assay of NK cells treated with ciliobrevin D (100 μM) or DMSO control. NK resistant Raji cells were used as innocent bystander cells. Compared to DMSO, cytotoxic function of YTS against 721.221 cells slightly increased after ciliobrevin D treatment (13D, black), whereas lysis of K562 cells by NK92 cells was not affected (13E, black). Bystander killing of Raji cells by YTS (13D, red) and NK92 (13E, red) cells both increased with ciliobrevin D treatment. Representative experiments from three independent experiments for YTS and NK92 cells were shown.

FIGS. 14A-14J. No effect of ciliobrevin D on the specific killing rate or viability of human NK cells. YTS or NK92 cells co-cultured with 51Cr-labeled Raji cells in the absence of 721.221 or K562 cells showed no spontaneous killing of the Raji cells (14A). Flow cytometry-based cytotoxicity assay of NK cells treated with Ciliobrevin D or DMSO vehicle control. With the susceptible targets alone, the cytotoxic functions of YTS (14B), NK92 (14C) and eNK (14D) cells against 721.221 and K562 cells were not affected after ciliobrevin D treatment as compared to DMSO control. The viability of YTS (14E, 14H), NK92 (14F, 14I) and eNK (14G, 14J) cells in the standard cytotoxicity assay and the bystander killing assays were not different between ciliobrevin D- and DMSO-treated groups. Representative experiments from three independent experiments for YTS and NK92 cells as well as three healthy donors for eNK cells were shown.

FIGS. 15A-15B. CAR-bearing cells alone and in conjugates with tumor targets show converged lytic granules in the presence of cytokines. Fixed cell confocal microscopy of CD19 CAR T, CD19 CAR NK, PSCA CAR T, GD2 CAR NKT, HER2 CAR T cells in conjugates with their respective tumor targets (15A, top panel) or alone (15B, bottom panel) in the presence of cytokine. Quantitative analyses of lytic granule distance from the MTOC are shown as a feature of the degree of granule convergence in the CAR-bearing therapy cells (15B).

FIG. 16. CAR-bearing cells in culture conditions have converged lytic granules. Fixed cell confocal microscopy of non-transduced and CAR-bearing cells in cytokine removed or normal culture conditions. Both non-transduced and CAR-bearing cells showed converged lytic granules in normal culture condition which contains the cytokines, IL-7 and IL-15, whereas in cytokine-removed culture condition, granules were dispersed in the cytoplasm. Quantitative analyses of lytic granule distance from the MTOC are shown as a feature of the degree of granule convergence in CAR-bearing cells.

FIG. 17. Ciliobrevin D can disperse lytic granules in CD19 CAR-bearing T cells. Live cell confocal microscopy of CD19 CAR-bearing T cells untreated or treated with ciliobrevin D (30 and 100 μM). Lytic granule positioning was tracked using LysoTracker Red. CAR-bearing T cells treated with ciliobrevin D showed dispersed lytic granules compared to the untreated cells. Quantitative analyses of lytic granule distance from the MTOC are shown as a feature of the degree of granule convergence in CD19 CAR-bearing T cells.

FIG. 18. Ciliobrevin D blocks stimulation-induced convergence in CD19 CAR-bearing T cells. Live cell confocal microscopy of CD19 CAR-bearing T cells treated with DMSO or ciliobrevin D (50 μM). Lytic granule positioning was tracked using LysoTracker Red. Ciliobrevin D-treated CD19 CAR-bearing T cells showed dispersed lytic granules either straight out of culture or 0.5 h post cytokine re-addition as compared to the DMSO control. The culture medium for CD19 CAR T cells contains IL-7 and IL-15. One hour after cytokine removal, lytic granules in the DMSO and ciliobrevin D-treated cells both showed dispersion. Quantitative analyses of lytic granule distance from the MTOC are shown as a feature of the degree of granule convergence in CD19 CAR-bearing T cells.

FIG. 19. Ciliobrevin D blocks stimulation-induced convergence in CD19 CAR-bearing NK cells. Live cell confocal microscopy of CD19 CAR-bearing NK cells treated with DMSO or ciliobrevin D (50 μM). Lytic granule positioning was tracked using LysoTracker Red. Ciliobrevin D-treated CD19 CAR-bearing NK cells showed dispersed lytic granules either straight out of culture or 0.5 h post cytokine re-addition as compared to the DMSO control. The culture medium for CD19 CAR NK cells contains IL-2. One hour after cytokine removal, lytic granules in the DMSO and ciliobrevin D-treated cells both showed dispersion. Quantitative analyses of lytic granule distance from the MTOC are shown as a feature of the degree of granule convergence in CD19 CAR-bearing T cells.

FIG. 20. Ciliobrevin D blocks stimulation-induced convergence in GD2 CAR-bearing NK cells. Live cell confocal microscopy of GD2 CAR-bearing T cells treated with DMSO or ciliobrevin D (50 μM). Lytic granule positioning was tracked using LysoTracker Red. Ciliobrevin D-treated GD2 CAR-bearing T cells showed dispersed lytic granules either straight out of culture or 0.5 h post cytokine re-addition as compared to the DMSO control. The culture medium for GD2 CAR T cells contains IL-7 and IL-15. One hour after cytokine removal, lytic granules in the DMSO and ciliobrevin D-treated cells both showed dispersion. Quantitative analyses of lytic granule distance from the MTOC are shown as a feature of the degree of granule convergence in GD2 CAR-bearing T cells.

FIG. 21. Bystander killing with increasing amounts of ciliobrevin D in the presence of CD19 CAR-bearing T cells. Standard 4-hour ⁵¹Cr cytotoxicity assay of CD19 CAR T cells treated with ciliobrevin D (25, 50 and 100 μM) or DMSO control. CD19-expressing Daoy cells (Daoy-CD19, a medulloblastoma cell line engineered to express CD19) were used as inciting target cells to trigger the activation of CD19 CAR-bearing T cells. BV173 cells, a human acute lymphoblastic leukemia cell line, were used as non-susceptible bystander cells to measure the degree of collateral damage caused by non-directional degranulation. The non-specific lysis of BV173 cells by CD19 CAR T cells showed dose-dependent increases at each effector to target ratio after ciliobrevin D treatment compared to the DMSO control.

FIG. 22. Bystander killing with increasing amounts of ciliobrevin D in the presence of CD19 CAR-bearing NK cells. Standard 4-hour ⁵¹Cr cytotoxicity assay of CD19 CAR NK cells treated with ciliobrevin D (12.5, 25, and 50 μM) or DMSO control. Daoy-CD19 cells were used as inciting target cells to activate CD19 CAR-bearing NK cells. Wildtype Daoy cells were used as innocent bystander cells to measure the degree of collateral damage caused by non-directional degranulation. The non-specific lysis of Daoy cells by CD19 CAR NK cells showed dose-dependent increases after ciliobrevin D treatment compared to the DMSO control.

FIG. 23. Bystander killing with ciliobrevin D in the presence of GD2 CAR-bearing T cells. Flow cytometry-based cytotoxicity assay of GD2 CAR-bearing T cells treated with ciliobrevin D (90 μM) or DMSO control. BV173 cells were used as non-susceptible bystander cells to measure the degree of non-specific killing (Left). Daoy-CD19 cells were used as activating target cells (Right). After 48 h incubation, percent cell lysis and total cell number were measured using SYTOX Blue viability dye and flow cytometer cell counting beads, respectively. The results were normalized to the DMSO control for demonstration. The non-specific lysis of BV173 cells increased after ciliobrevin D treatment while specific lysis of Daoy-CD19 cells was not affected. Moreover, after 48 h incubation, the total number of BV173 (bystander) cells decreased by ˜20% with ciliobrevin D treatment, whereas that of the Daoy-CD19 (target) cells remained similar to the DMSO control.

FIG. 24. Converged lytic granules in a DMSO-treated CD19 CAR T cell conjugated with a Daoy-CD19 cell. Live cell confocal microscopy of a DMSO-treated CD19 CAR T cell conjugated with a Daoy-CD19 target cell. Cell mixtures were imaged every 90 sec for 60 min. Time point 1 (T1) represents the time when imaging started. Target cell death occurred quickly at T5 (7.5 min) as indicated by uptake of SYTOX Blue viability dye and loss of adherence. Lytic granules in the CD19 CAR T cell remained converged throughout the video, from effector-target conjugate formation to target death. Red, LysoTracker Red (lytic granules); blue, SYTOX Blue viability dye; green, CD19 CAR.

FIG. 25. Dispersed lytic granules in a ciliobrevin D-treated CD19 CAR T cell conjugated with a Daoy-CD19 cell. Live cell confocal microscopy of a ciliobrevin D-treated CD19 CAR T cell conjugated with a Daoy-CD19 target cell. Cell mixtures were imaged every 90 sec for 60 min. Time point 1 (T1) represents the time when imaging started. Target cell death occurred rapidly at T5 (7.5 min) as indicated by uptake of SYTOX Blue viability dye and loss of adherence. Lytic granules in the CD19 CAR T cell remained dispersed throughout the video, from effector-target contact formation to target death. Red, LysoTracker Red (lytic granules); blue, SYTOX Blue viability dye; green, CD19 CAR.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method or composition described herein can be implemented with respect to any other method or composition described herein.

### I. General Embodiments

In a number of particular cell types, lytic granules are a specialized secretory organelle that comprise particular secretory proteins that function to destroy other cells. These specialized lysosomes can destroy whole cells as a consequence of their secretion. Although under normal conditions in vivo, lytic granules secreted from a particular cell converge to target a single cell for destruction, the present disclosure allows for modulation of that process to instead impart release of lytic granules in a disperse, non-converged manner, thereby killing multiple cells.

In particular embodiments of the disclosure, cytotoxicity by certain immune cells is mediated by tightly regulated binary degranulation events. Lytic granule convergence promotes directed degranulation that prevents bystander killing. Therefore, blocking convergence promotes bystander killing, so in particular embodiments of the disclosure, therapy for a medical condition in which cellular death is beneficial employs use of one or more agents that block convergence. In particular embodiments, methods of the disclosure employ promotion of non-directional degranulation, such as via dispersion, to increase bystander killing in cellular environments, such as tumor environments.

In particular cases the methods and compositions of the disclosure directly target one or more steps of a pathway that allows lytic granule traffic to the microtubule organizing center (MTOC). In an initial step of an exemplary pathway, an NK cell (as an example of a lytic granule-comprising cell) recognizes a target cell, and the dynein/dynactin complex transports the lytic granules to the MTOC. Next, the lytic granules converge to the MTOC independently of microtubule dynamics or actin reorganization at an immunological synapse (IS) between the NK cell and the target cell. Finally, the MTOC gradually polarizes along with the lytic granules to the IS where their contents are directed onto the target cell.

The present disclosure manipulates such a pathway by targeting one or more steps of the pathway. Such an embodiment provides compositions for enhancing cellular immunotherapies to prevent convergence of the granules and avoid converged release of granule contents upon action of the cells in vivo.

In other embodiments, the compositions themselves are delivered in vivo as an adjunct therapy to a cancer therapy.

### II. Compositions Related to Lytic Granule Position Modulating Agents

The present disclosure concerns compositions that comprise one or more agents that impact the movement and/or activity of lytic granules in cells, such as agents that inhibit convergence of lytic granules in the cells, control positioning of lytic granules in the cells, or maintain lytic granules near the surface of the cells (for example, within one micron to the closest edge of a lytic granule) and away from the MTOC, for example. Such agents may be referred to as lytic granule position-modulating agent(s). In some cases, one or more lytic granule/cell-modulating agents are used together, although in other cases a single lytic granule position-modulating agent is utilized.

The lytic granule position-modulating agent(s) may be of any kind so long as it allows disperse release of lytic granules from cells that naturally comprise lytic granules. In specific embodiments, the agent modulates movement of the granules within the cell, such as to prevent their convergence at a focalized point or region within the cell. In specific embodiments, the agent inhibits or impedes movement of the granules within the cell, so long as the cell is still able to release lytic granule contents at least in part (if the release is partially inhibited by an agent, in specific cases it is still useful if it allows for non-directional degranulation). The lytic granule/cell-modulating agent may be one or more of the following: a) an inhibitor of a motor protein involved in transport of the granules, b) an inhibitor of an activating receptor of the motor protein; c) an inhibitor of a signaling molecule for the motor protein; d) an inhibitor of a receptor that induces a signaling molecule for the motor protein function; d) an inhibitor of a molecule linking lytic granules to microtubules and/or motor proteins; f) expression or overexpression of a molecule in a cytotoxic cell that interferes with or eliminates dynein (for example, p50/dynamitin and CC1 domain of p150^{Glued}); and/or g) an agent that eliminates the expression of a protein that facilitates granule convergence. For agents that eliminate the expression of a protein that facilitates granule convergence, the agent may be eliminate expression of RASGRP1, dynein, dynactin, HkRP3, Rab7, RILP, ORP1L, Pyk2, CLP170, leupaxin, LFA1, CD11a, CD18, CD54. Src, NIK, RASGRP1, PTEN, ILK, PINCH1, γ-parvin, paxillin, RhoGEF7; CDC42, Par6, aPKC, GSK3β, APC, IQGAP1, CLIP-170, Arl8b, or a combination thereof, for example.

In certain cases, the lytic granule position-modulating agent is an inhibitor of one of more members of pathways involved in cell-mediated cytotoxicity, including, for example, a dynein/dynactin pathway. Thus, in specific cases, the lytic granule position-modulating agent(s) are one or more of the following: a) an inhibitor of dynein; b) an inhibitor of an activating receptor of dynein; c) an inhibitor of a signaling molecule for dynein function; and/or d) an inhibitor of a receptor that induces a signaling molecule for dynein function.

In specific embodiments, the lytic granule position-modulating agent is an inhibitor of dynein; one or more of the dynactin complex proteins (comprising three major structural domains: (1) sidearm-shoulder: DCTN1, DCTN2/dynamitin, DCTN3/p22/p24; (2) the Arpl rod: Arpl/centractin, actin, CapZ; and (3) the pointed end complex: Actr10/Arp11, DCTN4/p62, DCTN5/p25, and DCTN6/p27); Rab7; RILP; ORP1L; Pyk2; CLP170; leupaxin; RhoGEF7; LFA1; CD11a; CD18; CD54; Src; NIK; RASGRP1; PTEN; ILK; PINCH1; γ-parvin; paxillin; CDC42; Par6; aPKC; GSK3β; APC; IQGAP1; CLIP-170; HkRP3; Arl8b; or one or more agents that otherwise links the lytic granules to microtubules or dynein including HkRP3; or a combination thereof. In specific cases, the inhibitor blocks an integrin or its signaling.

In particular embodiments, the inhibitor is an dynein inhibitor molecule, such as one or more ciliobrevins or functional derivatives thereof; in one case the ciliobrevin is Ciliobrevin D (also known as 2-(7-chloro-4-oxo-3,4-dihydroquinazolin-2(1H)-ylidene)-3-(2,4-dichlorophenyl)-3-oxopropanenitrile). Examples of anti-dynein antibodies include at least 74.1 (ThermoFisher Scientific; Waltham, Mass.) and ab111177 (Abeam; Cambridge, Mass.).

The inhibitor may be a protein, peptide, nucleic acid, small molecule, or combination thereof. In certain cases, the inhibitor comprises the CC1 domain of P150^{glued} of dynactin (the dynein intermediate chain) or the p50 dynamitin protein. In certain embodiments, the inhibitor is an antibody of any kind (polyclonal, monoclonal, chimeric, humanized, human, etc.), a fragment of an antibody (Fab, single chain variable fragment (scFv), single domain antibody fragment, etc.), a soluble ligand or receptor, a cell permeable peptide, or a mixture thereof. In a specific embodiment, the antibody is an anti-LFA-1 antibody or fragment thereof, an anti-CD18 antibody, an antibody to CD11a, or a combination thereof. In cases wherein the inhibitor is a nucleic acid, the nucleic acid may be a vector (viral or non-viral), such as one that expresses a shRNA, siRNA, miRNA, coding RNA, and so forth. In specific embodiments, a cell that is treated with a lytic granule position-modulating agent may be modified by the CRISPR/Cas9 gene deletion system. In cases wherein the inhibitor is a nucleic acid, the nucleic acid may be provided exogenously to the cells of the cellular therapy, or the nucleic acid may be expressed within the cells of the cellular therapy. In specific cases, a nucleic acid that encodes an inhibitor (which may be the resultant expressed nucleic acid or may be the ultimate translated protein product) is comprised on a vector within the cells of the cellular therapy such that its expression produces the desired inhibitor. The vector may or may not integrate into the genome of the cells of the cellular therapy. When the cells of the cellular therapy express another molecule that enhances their activity (such as an engineered receptor (chimeric cytokine receptor or chimeric antigen receptor, for example), the nucleic acid that encodes the inhibitor and the nucleic acid that encodes the other molecule may or may not be present on the same nucleic acid molecule. In cases wherein they are on the same nucleic acid, in specific cases they are regulated by different regulatory regions; they may be separated by an IRES or 2A in some cases.

In specific embodiments, the one or more lytic granule/cell-modulating agent(s) inhibit activity and/or expression of a particular component of a pathway related to granule movement or positioning.

When exposed to cells and/or delivered to an individual in need thereof, the lytic granule/cell-modulating agent(s) may be provided in an excipient or carrier.

The lytic granule position-modulating agent(s) may be commercially obtained or otherwise produced by standard methods in the art.

### III. Methods of Use of Lytic Granule Position-Modulating Agents

In particular embodiments, lytic granule position-modulating agents are utilized for the killing of surrounding cells that might otherwise be specifically protected. In embodiments of the disclosure, one or more lytic granule position-modulating agent(s) are utilized to modulate at least one aspect of lytic granules in cells or secretion therefrom. In a particular embodiment the agent(s) affect granule positioning to tailor cytotoxicity and/or otherwise promote effectiveness for therapeutic approaches using cytotoxic cells.

In particular embodiments, the lytic granule position-modulating agent(s) are employed to modulate the lytic granules such that convergence of the granules is inhibited or impeded, or that the positioning of lytic granules is otherwise controlled in order to promote their dispersion. In certain cases the lytic granule position-modulating agent(s) are utilized to improve a treatment that causes cytotoxicity to cells, such as cellular therapy, including for cancer. Another example includes therapeutic antibodies, such as anti-cancer antibodies including monoclonal antibodies (for example an antibody against CD20 (Rituximab and Ibritumomab); Alemtuzumab against CD52; and Atezolizumab against PD-L1). In certain embodiments, extracelluar infection or pathogenic (viral, bacterial, fungal) infection of specific tissues is treated. In particular cases, the one or more lytic granule position-modulating agent(s) are not delivered to the individual themselves but instead are utilized to improve a therapy that is to be delivered to the individual.

In some cases, the cells being modified are immune cells (T cells, NK cells, NK T cells, cytotoxic innate lymphoid cells, or a mixture thereof) or cytotoxic cells (for example, cells from NK92 cell line). Any cells for the cellular therapy may or may not be from a cell line. The cells may or may not be obtained from an individual that is being treated. Thus, the cells may be autologous or allogeneic.

In particular embodiments, the one or more lytic granule position-modulating agent(s) are exposed to the cells to be modified in a sufficient amount and under suitable conditions and duration, and this occurs prior to delivery of the cells to the individual. The exposure of the cells by the agents occurs ex vivo or in vitro, in specific embodiments. In at least some cases, the cells are exposed to the one or more lytic granule position-modulating agent(s) within minutes, hours, or days of delivering the cells to the individual. However, in certain cases the cells are treated and then cryopreserved and stored for week, months, or years in that state prior to use. Such cells represent so-called “off the shelf” cellular therapeutics. In doing so, the cells using methods of the disclosure are treated and then cryopreserved until they could be taken advantage of in therapy. In some cases, the exposure of the cells to the one or more lytic granule position-modulating agent(s) is at least or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes, or is at least or no more than 1, 2, 3, 4, 5, 6, 7, 8, 9, 10, 11, 12, 13, 14, 15, 16, 17, 18, 19, 20, 21, 22, 23, or 24 hours, or is at least or no more than 1, 2, 3, 4, 5, 6, or 7 days. The maximum time that the cells are exposed to the agent is the entire culture expansion of the cells, although in specific cases to avoid difficulties in their division and further proliferation (for example, in the case of a dynein inhibitor), the maximum time may be about 30 minutes, including at least 30 minutes. In specific cases, the concentration of the agent is 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, or 100 μm, as examples. A range of concentration may be 5 μm-100 μm, 5 μm-50 μm, 5 μm-25 μm, 10 μm-100 μm, 1 μm-75 μm, 10 μm-50 μm, 25 μm-100 μm, 25 μm-75 μm; 25 μm-50 μm; 50 μm-100 μm; 50 μm-75 μm; or 75 μm-100 μm. The inhibitor may be prepared as a stock solution in DMSO, in specific cases. In other cases, an agent such as ciliobrevin D may be administered at a concentration on the order of 1, 5, 10, 15, 20, 25, 30, 40, 50, 75, or 100 mM, for example.

In one example, cells to be delivered as part of a therapy are exposed in a vessel (a dish, flask, well, plate, and so forth) to a sufficient amount of an inhibitor, such as ciliobrevin D, for a sufficient time and conditions such that the cells are modified for lytic granule movement and/or position. The modification for the cells may manifest prior to and/or following delivery. Once the cells are sufficiently exposed, a therapeutically effective amount of the cells are provided to the individual in need of such therapy. In some cases, one or more of the lytic granule position-modulating agent(s) are provided to the individual in addition to or alternative to being a modifier of the cells of cellular therapy.

In specific embodiments, one can treat the cells of the cellular therapy with ciliobrevin D or an antibody against LFA1 (for example, an anti LFA1 blocking antibody Fab) to alter granule positioning prior to their delivery (such as by infusion). The cells may be delivered by direct injection into a site of disease.

In particular embodiments, the cells of the cellular therapy are modified, such as modified to express non-natural receptors on the surface of the cell. In specific embodiments, the receptor is a chimeric antigen receptor (CAR), and the CAR may be first generation, second generation, third generation, and so on. The CAR may target any antigen, including any tumor antigen, and the CAR may be configured to target one or more than one antigens. In specific embodiments, the antigen is 5T4, αᵥβ₆ integrin, B7-H3, B7-H6, CAIX, CD19, CD20, CD22, CD30, CD33, CD44, CD44v6, CD44v7/8, CD70, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, HLA-A1+MAGE1, HLA-A1+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, Survivin, TAG72, TEMs, VEGFR2, targets of therapeutic monoclonal antibodies, and so forth.

### IV. Adjunct Therapy

In some embodiments, the one or more lytic granule position-modulating agent(s) are provided to an individual in need thereof as a therapy, as opposed to other embodiments addressed herein wherein the agent(s) are not the therapy but instead improve a therapy.

In particular embodiments, there are methods of treatment and methods of enhancing a therapy for cancer in an individual, both comprising the step of administering to the individual an effective amount of one or more agents that inhibits convergence of lytic granules in immune cells of the individual. In at least some cases, the individual is provided a therapeutically effective amount of another cancer therapy in addition to the one or more lytic granule position-modulating agent(s). In cases wherein a second therapy to the one or more lytic granule position-modulating agent(s) may be given to the individual, the second therapy may be of any kind, including another inhibitor of an undesirable protein. In specific cases, the second therapy is a drug, hormone therapy, immunotherapy, and so forth.

One or more lytic granule position-modulating agent(s) may be delivered as a therapy to an individual with cancer, in certain cases, and the delivery route may be by any route, such as systemic or local, including infusion, intravenous, intrarterial, topical, subdermal, oral, nasal, subcutaneous, transdermal, and so forth.

In particular embodiments, the lytic granule position-modulating agent(s) is an antibody that blocks motor protein function or is an antibody that blocks the function of an activating receptor of the motor protein. Such agents prevent granule convergence, and in specific embodiments may or may not be given to an individual in addition to an anti-cancer targeting antibody, such as rituximab. This would allow for the cells encountering such cancer (or other disease) targeting antibody to perform diffuse killing of local cells and not just activity directed against the single diseased cell.

### V. Pharmaceutical Preparations

In some cases, one or more lytic granule position-modulating agent(s) are exposed to cells for cellular immunotherapy (for example, for “treatment of a treatment”) or they may instead be provided to an individual as part of a treatment themselves.

Pharmaceutical compositions of the present invention comprise an effective amount of one or more lytic granule position-modulating agent(s) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one lytic granule/cell-modulating agent will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

Compositions comprising lytic granule position-modulating agent(s) may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The lytic granule position-modulating agent(s) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include lytic granule position-modulating agent(s) and an aqueous solvent, although in certain cases the composition comprises a lipid. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the lytic granule/cell-modulating agent(s) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the lytic granule position-modulating agent(s) are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof, a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, lytic granule position-modulating agent(s) may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound lytic granule position-modulating agent(s) may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VI. Kits of the Disclosure

Any of the compositions described herein may be comprised in a kit. In a non-limiting example, a lytic granule position-modulating agent(s) and/or cells may be comprised in a kit. The kits will thus comprise, in suitable container means, a lytic granule/cell-modulating agent(s) and optionally one or more additional agents of the present invention.

The kits may comprise a suitably aliquoted lytic granule position-modulating agent(s) composition(s) of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the lytic granule/cell-modulating agent(s) and any other therapeutic and/or reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

In specific cases, the kit comprises a cancer therapy, including cells (such as the cells to be modified), chemotherapy, hormones, or combinations thereof.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Nk Cells Converge Lytic Granules to Promote Cytotoxicity and Prevent Bystander Killing

It was considered that one could increase efficiency of killing while minimizing bystander killing. To identify utility in lytic granule convergence in NK cell cytotoxicity, approaches were used to regulate the balance of signaling through LFA-1 and CD16 to either promote degranulation without granule polarization or both polarization and degranulation. This was combined with highly resolved 4-dimensional confocal and ultrasound-guided-acoustic-trap-microscopy (UGATm) system (Christakou et al., 2013) in order to create specific coordinated cell interactions and track lytic granules in live NK cells to correlate lytic granule positioning with target cell death. NK cell lytic granule convergence improves the efficiency of targeted lytic granule secretion and prevents bystander killing.

CD16 Engagement Induces Conjugate Formation and Degranulation but not Lytic Granule Convergence in NK Cells

NK cells converge lytic granules upon recognition of target cells. Although the signals directing this process have been elucidated, it remains unclear as to why NK cells converge granules. To address this question, a Drosophila S2 target cell system was utilized and modified. Because S2 cells are evolutionarily distant from mammalian cells, they do not express ligands recognized by human NK cells and are therefore inert (March et al., 2010). Expression of human intercellular adhesion molecule (ICAM-1) on S2 cells ligates NK cell LFA-1 and causes lytic granule polarization without degranulation, whereas opsonization of the S2 cell with antiserum engages the IgG Fc receptor CD16 and promotes degranulation without granule/MTOC polarization (Bryceson et al., 2005). The inventors desired to determine precise granule positioning after isolated triggering of either receptor to better understand potential directionality in granule targeting.

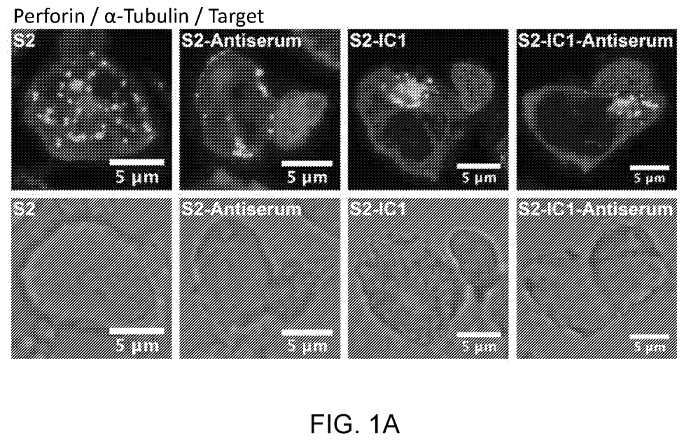

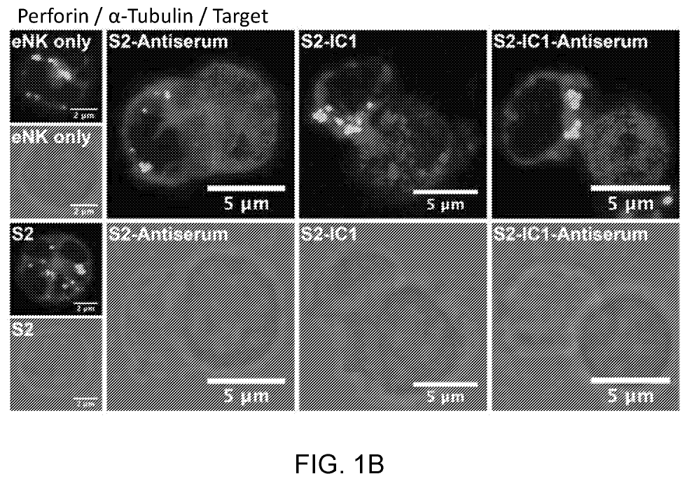

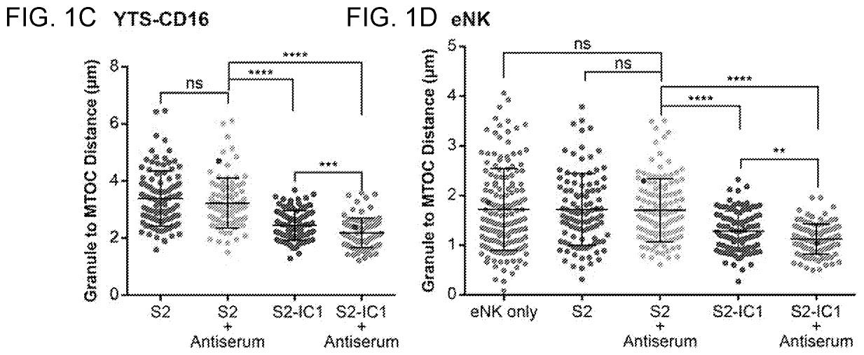

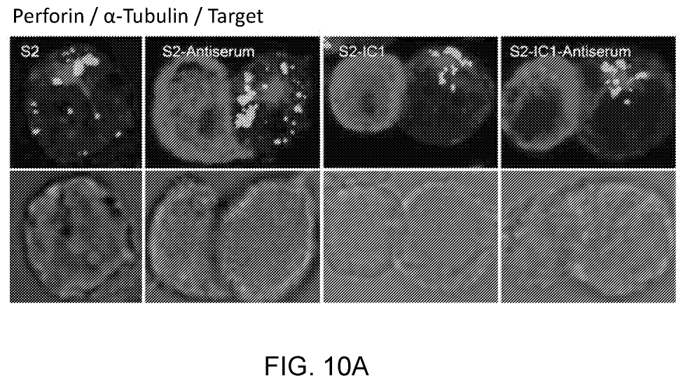

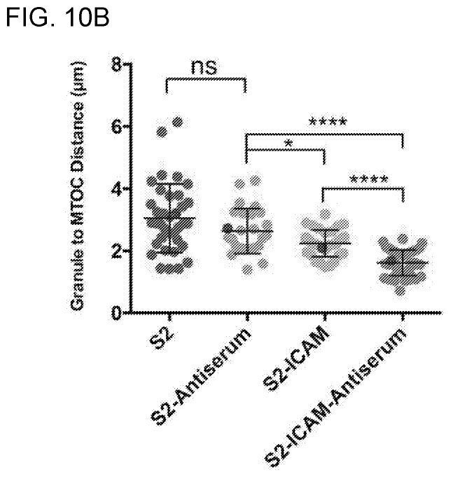

Two different CD16-expressing clonal human NK cell lines (YTS and NK92) were used, as they are effective in cytotoxicity but are amenable to in vitro manipulation as well as ex vivo NK (eNK) cells, which more directly reflect actual human immunity. Of the cell lines, the lytic granule positioning in YTS cells is likely more reflective of eNK cells, as NK92 cells require growth in IL-2, which can independently alter granule positioning via Src signaling (James et al., 2013). In each type of NK cell, the distance was measured between every lytic granule and the MTOC (in the plane of the MTOC) in NK cells incubated with S2 cells, S2 cells expressing the ligand for LFA-1-ICAM-1 (S2-IC1), or S2 or S2-IC1 cells coated with S2 antiserum to trigger CD16 (S2-Antiserum or S2-IC1-Antiserum). Lytic granules in CD16-expressing YTS (YTS-CD16, FIG. 1A, C) cells, NK92 (NK92-CD16, FIG. 10A, B) cells and eNK (FIG. 1B, D) cells converged to the MTOC when conjugated with S2-IC1 or S2-IC1-Antiserum cells. Lytic granules in NK cells incubated with S2-Antiserum cells, however, were not significantly different in their distance from the MTOC compared to those incubated with control S2 cells, or in unconjugated NK cells (FIGS. 1 and 10).

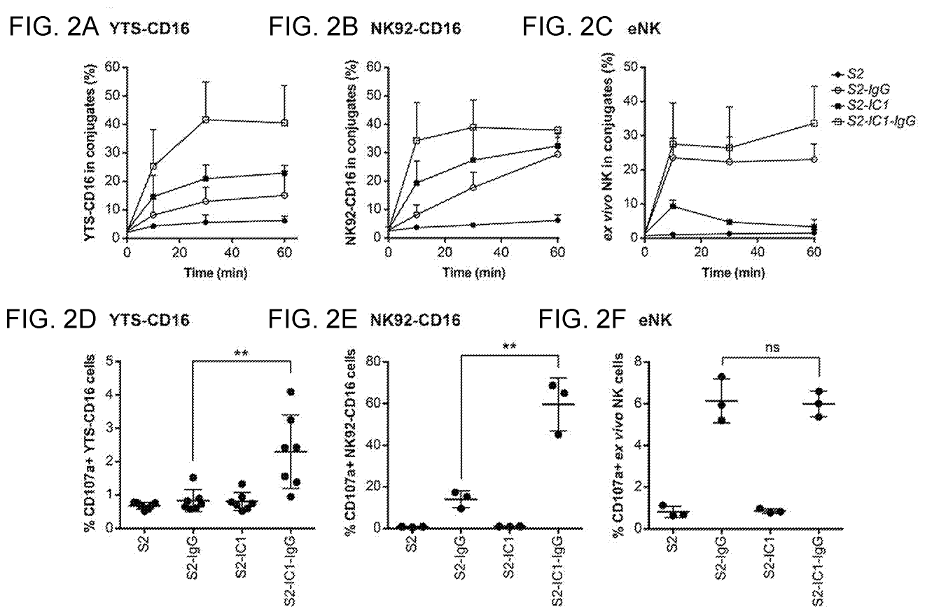

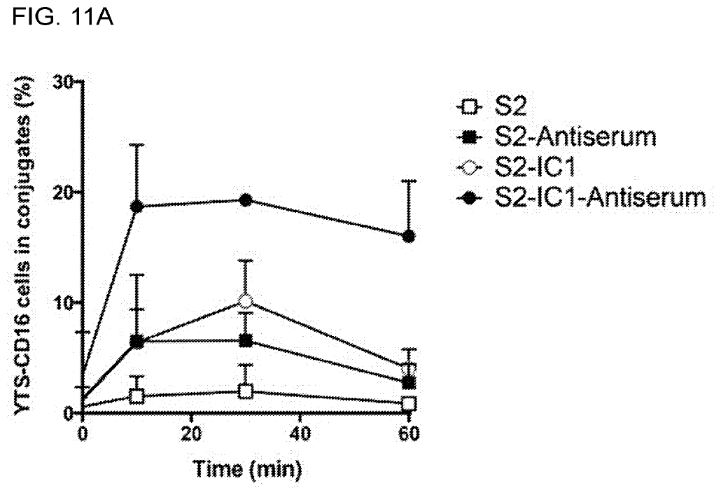

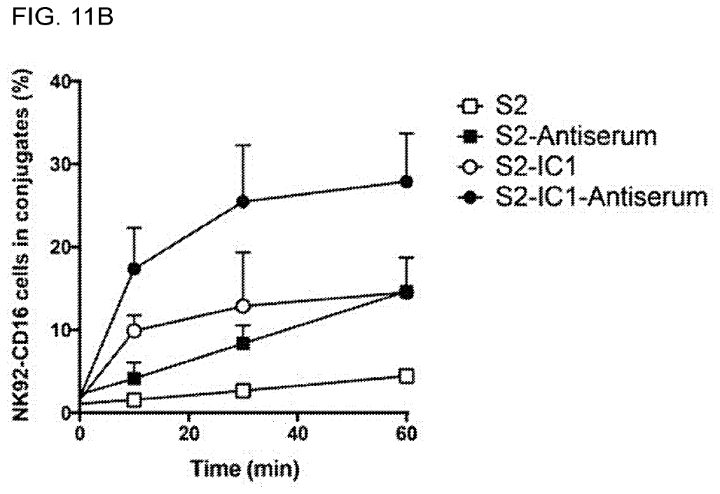

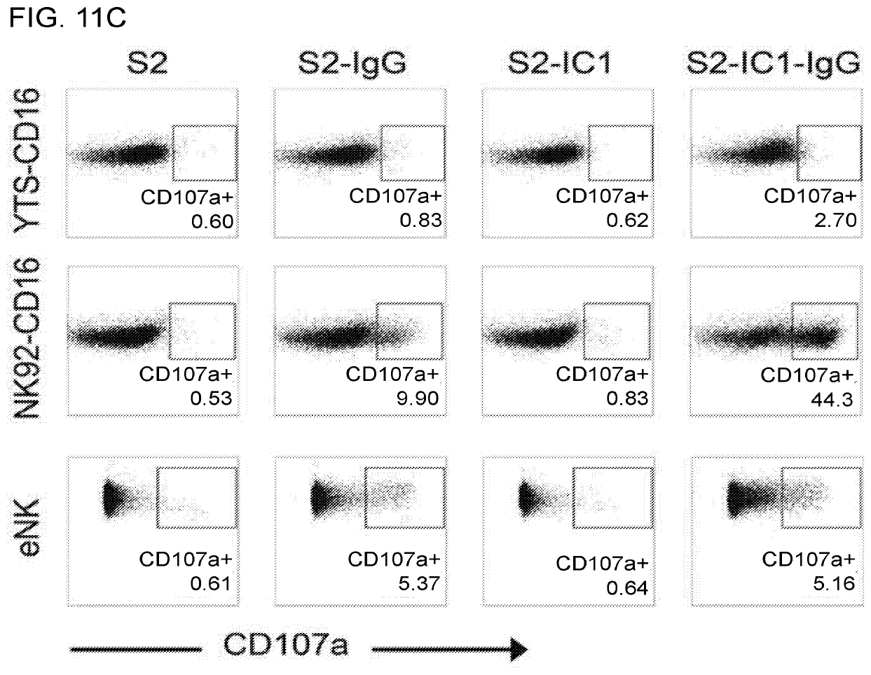

NK cell cytotoxicity consists of a stepwise series of tightly regulated cellular events that requires their contact with and adherence to target cells as an early step in the process (Mace et al., 2014). Although NK cells form conjugates with S2 cells coated with S2 antiserum (FIG. 11 A, B) (Bryceson et al., 2005), it was considered that purified anti-S2 IgG should perform similarly while potentially increasing the specificity of the signal input for NK cells via CD16 as any other serum components would be removed. Thus, the inventors developed an S2-specific IgG and used this to opsonize S2 or S2-IC1 (denoted as S2-IgG or S2-IC1-IgG), which were incubated with YTS-CD16, NK92-CD16, or eNK cells, for 0, 10, 30 and 60 min (unlabeled S2, or S2-IC1 cells were used as controls). CD16 engagement by anti-S2 IgG induced conjugate formation in YTS-CD16 cells (FIG. 2A), NK92-CD16 cells (FIG. 2B) and eNK cells (FIG. 2C), whereas conjugate formation with unlabeled S2 cells was negligible. In all NK cells tested, purified IgG allowed for comparable conjugate efficiency compared to antiserum.

To ensure that the purified anti-S2 antibodies trigger degranulation of the CD16-expressing NK cell lines and eNK cells, CD107a exposure was quantified on the NK cell surface using flow cytometry. YTS-CD16, NK92-CD16 or eNK cells mixed with S2, S2-IgG, S2-IC1 or S2-IC1-IgG cells demonstrated that engagement of CD16 induced degranulation. Addition of ICAM-1 further enhanced degranulation in the NK cell lines (FIG. 2D, E and FIG. 11C), but not in eNK cells which had similar CD107a levels when conjugated with S2-IgG or S2-IC1-IgG (FIG. 2F and FIG. 11C) as previously reported for antiserum-coated S2 cells (Bryceson et al., 2005). NK cells conjugated with S2 or S2-IC1 cells displayed negligible CD107a levels. Thus, purified anti-S2 IgG induced conjugate formation in the absence of ICAM-1 and triggered degranulation.

Convergence Promotes Lytic Granule Directed Secretion—

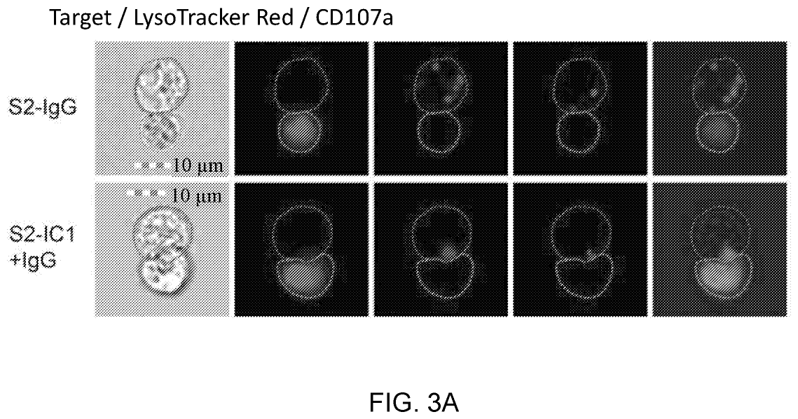

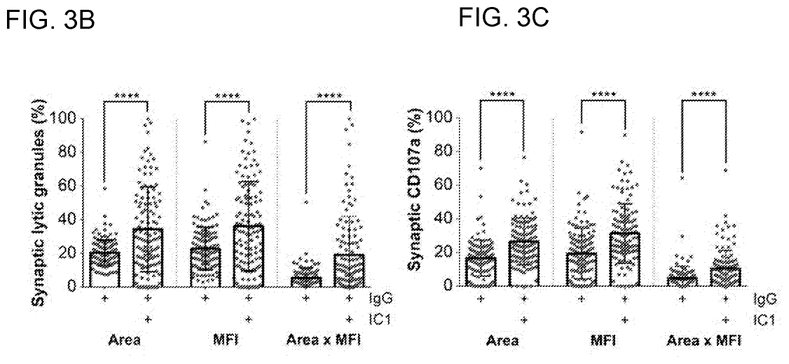

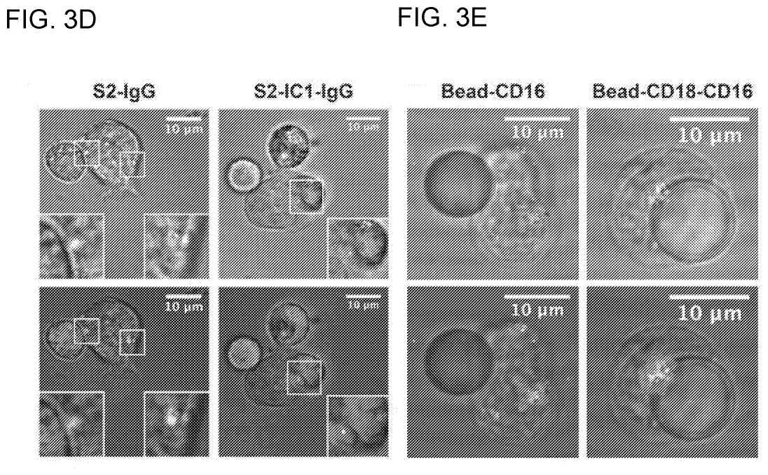

Because isolated ligation of CD16 promotes degranulation despite granules being diffusely localized, it was considered that at least some degranulation would be non-directional. Using imaging flow cytometry for high throughput measurement of NK cell-target cell conjugates, the inventors evaluated the positioning of degranulation relative to the immunological synapse (IS) in YTS-CD16 NK cells triggered with S2-IgG or S2-IC1-IgG (FIG. 3A). The area, mean fluorescence intensity (MFI) and total fluorescence of lytic granules (denoted by LysoTracker Red, FIG. 3B) and degranulation (denoted by CD107a, FIG. 3C) at the IS were significantly higher in YTS-CD16 cells conjugated with S2-IC1-IgG cells compared to those conjugated with S2-IgG cells. The latter displayed both lower synaptic granules and degranulation suggesting non-directionality despite the presence of an opsonized target cell.

To further define this process, live cell microscopy was performed to track the lytic granule movements while identifying degranulation events using a stably expressed degranulation indicator fusion protein (LAMP1-pHluorin) (Rak et al., 2011). While not amenable to introduction into eNK cells without inducing their activation, YTS-CD16 cells stably expressing LAMP1-pHluorin were generated. In these cells, acidified granules can be tracked via LysoTracker Red fluorescence with their release marked by a transition to green fluorescence due to granule de-acidification and pHluorin excitation. When these cells were conjugated with S2-IgG cells, degranulation events occurred outside of the IS (FIG. 3D, left), while with S2-IC1-IgG cells, degranulation was focused to the IS (FIG. 3D, right). YTS-CD16-LAMP1-pHluorin cells were conjugated to polystyrene beads coated with either anti-CD16 or anti-LFA-1+anti-CD16 and found that the former led to diffuse degranulation while the latter led to degranulation focused to the bead (FIG. 3E). Thus, NK cells activated by both CD16 and LFA-1 converged their granules and demonstrated highly focused synaptic degranulation, whereas those activated by CD16 did not, leading to non-directional degranulation.

Directed Secretion of Lytic Granules Promotes Specific Killing of the Targeted Cell—

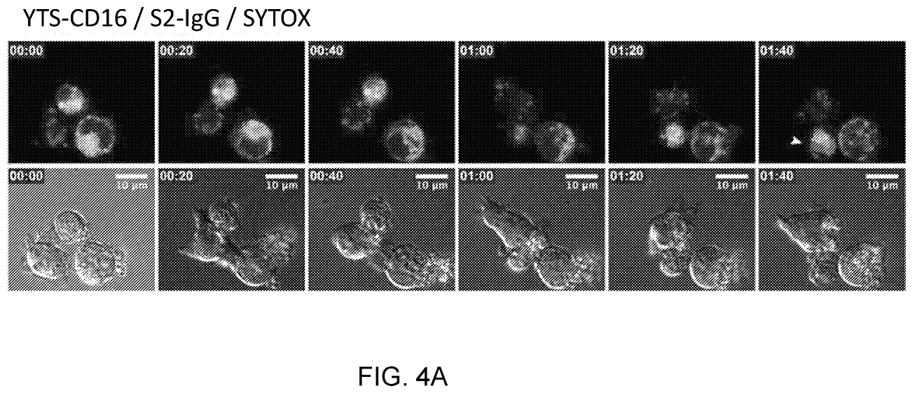

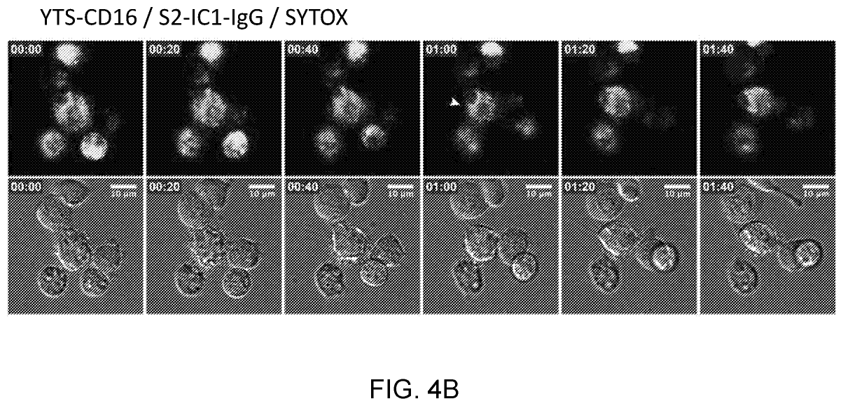

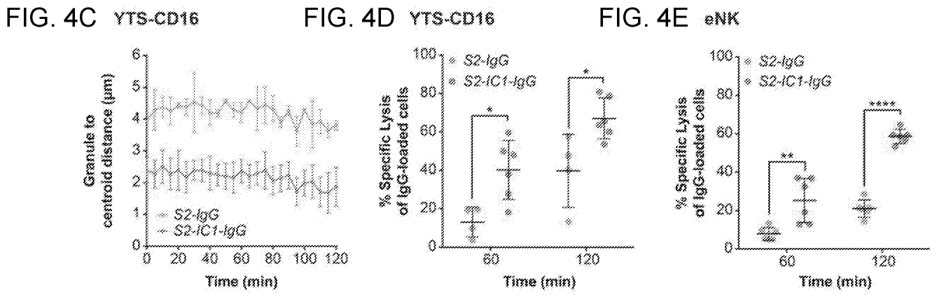

To understand how directed versus non-directed degranulation might contribute to efficiency of NK cell cytotoxicity, it was desirable to simulate tissue environments by creating aggregates of NK cells with various types of potential target cells that could be imaged by confocal microscopy to both track the movement of the lytic granules and quantify target cell death in real time. An UGATm system was utilized to create clusters of cells on demand, thus allowing us to exert control over the initiation of cell contact and signaling (Christakou et al., 2013). Viability of S2 cells and NK cells were not affected using the UGATm system (data not shown; (Ohlin et al., 2015)). NK cell lytic granules were tracked using LysoTracker Red and cell death detected by uptake of SYTOX Blue viability dye. Specific target cells were identified by pre-loaded vital dye. When YTS-CD16 cells were aggregated with S2-IgG cells, lytic granules remained diffusely distributed in the cytoplasm (FIG. 4A). When the same was performed with S2-IC1-IgG cells, the granules were highly converged and were polarized towards a target cell (FIG. 4B). The mean granule distance from the centroid was measured over time, showing that within 2-hour intervals, lytic granules in YTS-CD16 cells aggregated with S2-IgG cells did not converge to the MTOC (FIG. 4C). In contrast those aggregated with S2-IC1-IgG cells had granules that were mostly converged by the time that imaging could begin (consistent with our prior observations (James et al., 2013; Mentlik et al., 2010)).

Killing efficiency was evaluated in these experiments and defined as the proportion of total target cells that assimilated viability dye. The killing efficiency over a given timeframe was significantly higher in YTS-CD16 cells aggregated with S2-IC1-IgG cells compared to S2-IgG cells (FIG. 4D). As over 2 hours, YTS-CD16 cells aggregated with S2-IgG or S2-IC1-IgG cells induced an average of ˜40% and ˜67% of killing, respectively. The same was true for eNK cells, as when aggregated with S2-IgG cells they demonstrated a significantly lower killing efficiency compared to that of S2-IC1-IgG cells: ˜25% and ˜59%, respectively (FIG. 4E). This distinction in the eNK cells is important, as the total degranulation induced by the two different target cells was equivalent (FIG. 2F). Thus directional degranulation triggered by the combined signals of CD16 and LFA-1 enhances the efficiency of target lysis by NK cells.

Non-Directed Degranulation Increases Bystander Killing—

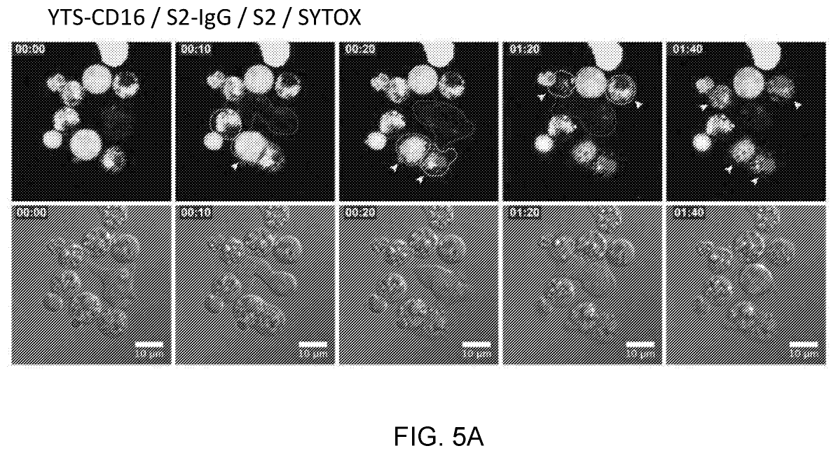

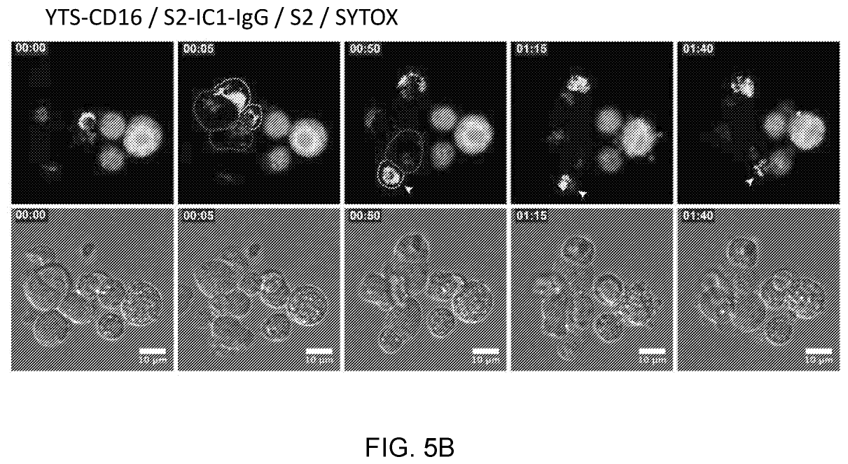

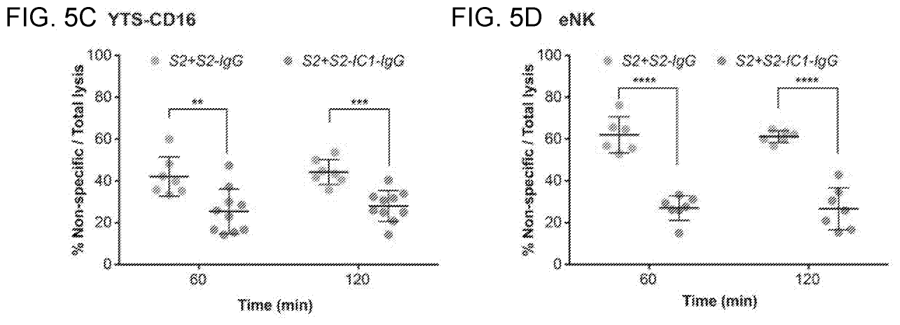

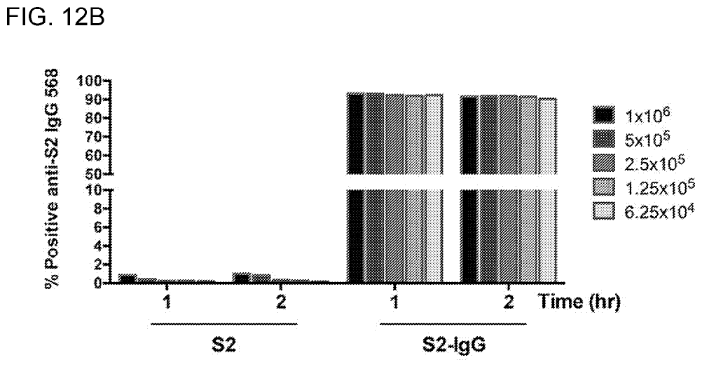

In a physiological environment, NK cells are challenged with a need to discern diseased cells from those that are healthy (Eriksson et al., 1999). It was considered that NK cells utilize precise positioning of their cytolytic machinery to promote accurate target cell killing without collateral damage. To determine if this precision was a feature of lytic granule convergence and directional degranulation, the UGATm experiments were performed with S2-IgG or S2-IC1-IgG cells but included differentially dye-labeled S2 cells. Thus yellow-labeled S2-IgG or S2-IC1-IgG target cells served as those capable of generating a signal for cytotoxicity, whereas green-labeled S2 cells served as “innocent bystanders”. Both granule positioning as well as death of the different target cells after aggregation with NK cells was measured. As identified in experiments with only one type of target cell, YTS-CD16 cells aggregated with S2-IgG cells contained dispersed lytic granules (FIG. 5A) whereas those aggregated with S2-IC1-IgG cells had highly converged granules polarized towards the target cells (FIG. 5B). The lysis of S2 cells was calculated as a proportion of total target cell lysis to determine the degree of bystander killing by NK cells in order to account for the fact that the overall killing of S2-IgG cells was lower likely as a feature of lesser signal input from this nonphysiologic target cell system. The rate of bystander killing significantly increased by ˜17% when YTS-CD16 cells were aggregated with S2 and S2-IgG cells compared to S2 and S2-IC1-IgG (FIG. 5C). To ensure the specificity of the target signal in these mixed cell experiments, the transfer of anti-S2 IgG from S2-IgG cells to plain S2 cells in the cell mixture was measured and was negligible (FIG. 12A, 12B). Thus directed secretion provided higher specificity in target killing. This effect was even more striking when eNK cells were aggregated with S2-IgG or S2-IC1-IgG with S2 bystander cells. Non-directed secretion triggered by CD16 signaling alone caused 35% higher bystander killing than directed secretion activated by co-engagement of CD16 and LFA-1 (FIG. 5D). Thus, convergence of lytic granules, which promotes directed degranulation therefore enables innocent bystander protection and allows for specificity of target killing.

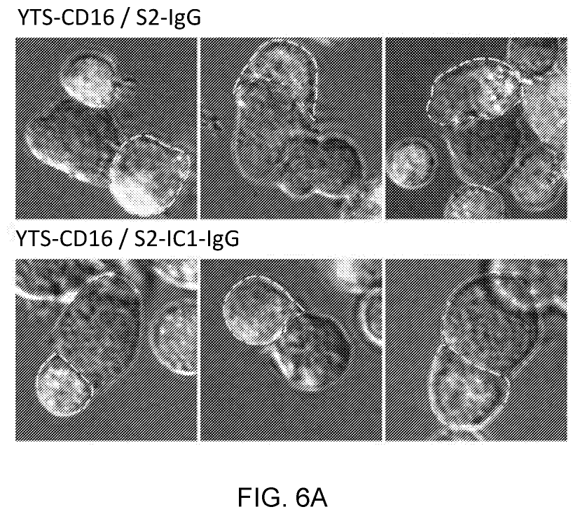

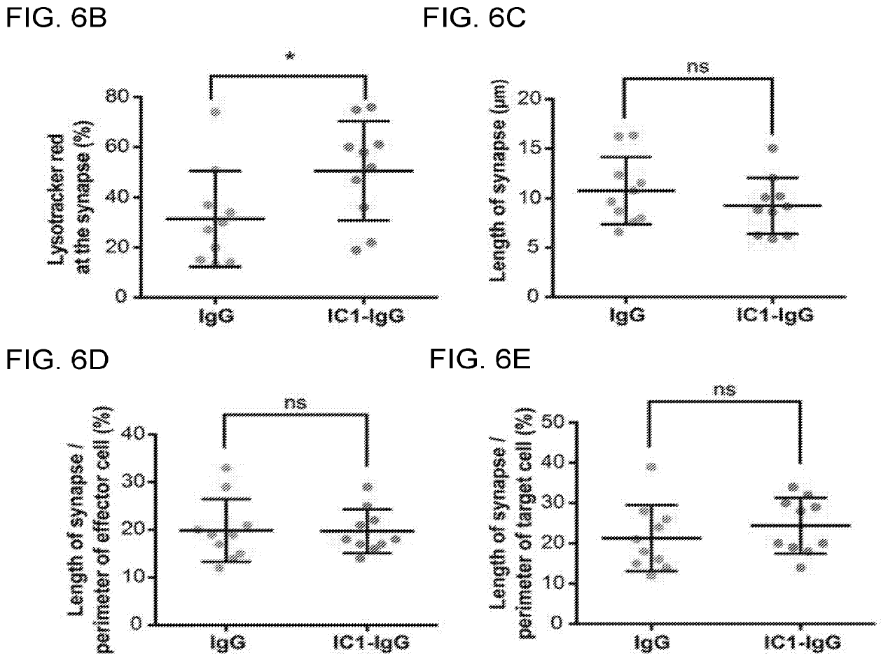

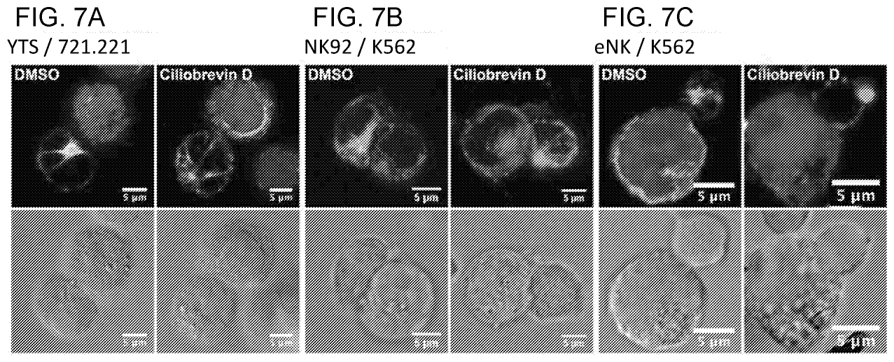

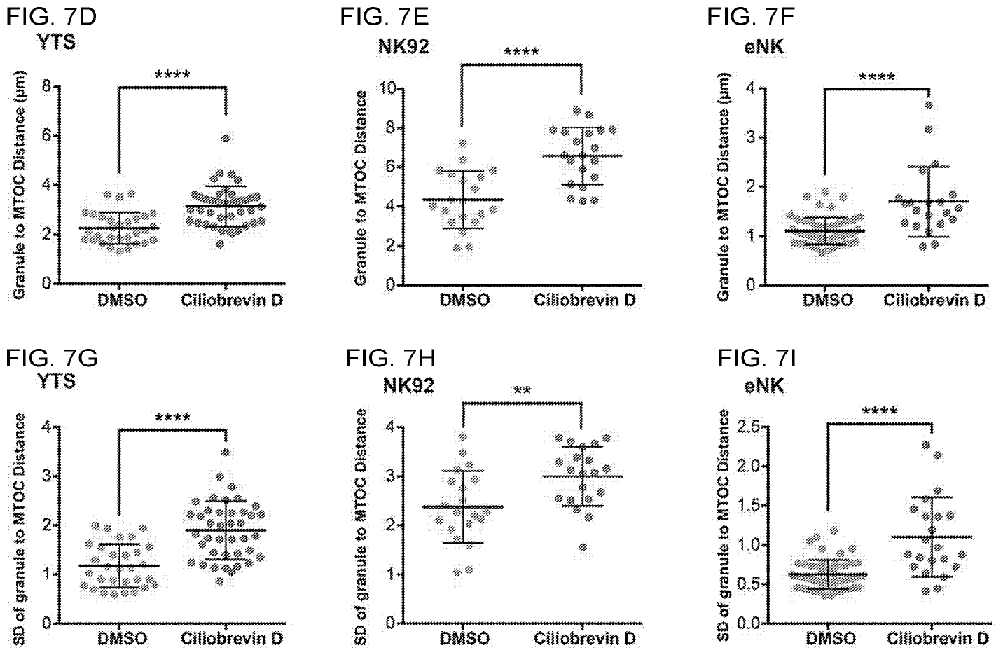

To evaluate whether the lack of LFA-1 signaling reduced potential “sealing” of the IS and hence promoted bystander killing by polarized degranulation events, the geometry of the synapse formed between YTS-CD16 cells and S2-IgG or S2-IC1-IgG cells was measured (FIG. 6A). While fewer lytic granules were at the synapse with the S2-IgG cells (FIG. 6B, consistent with FIG. 3B), the length of the synapse was not different from that formed with S2-IC1-IgG cells (FIG. 6C). Furthermore, the length of the IS as a feature of the perimeter of both the effector (FIG. 6D) and target (FIG. 6E) cell was also not different between the S2-IgG and S2-IC1-IgG cells. This suggests that the contact is not partial in S2-IgG and that it is indeed the non-directed release of granules away from the target that promotes bystander destruction.

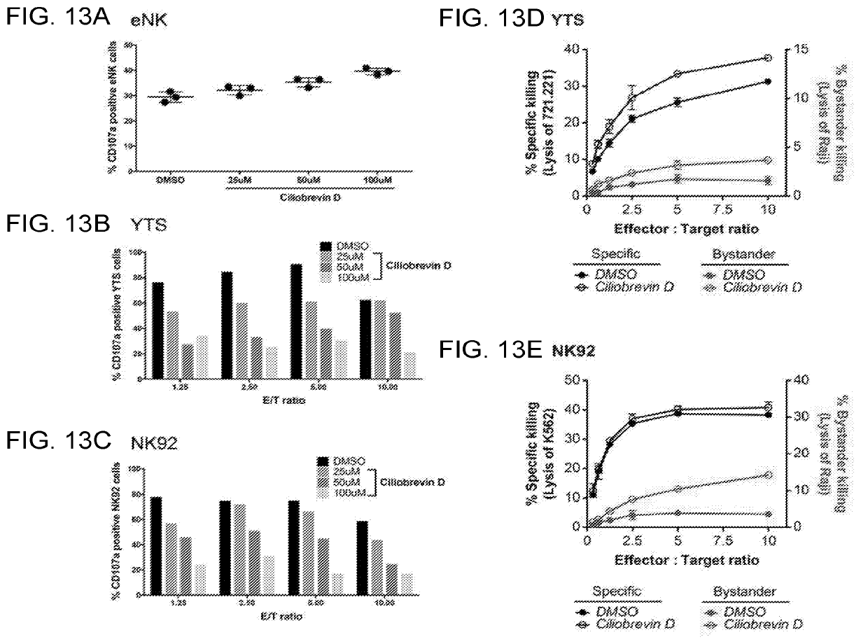

In previous studies, it was shown that granule convergence is dynein-dependent (Mentlik et al., 2010). To further characterize this, regarding the role for convergence in promoting killing efficiency and protecting bystanders, a small molecule dynein inhibitor ciliobrevin D (Firestone et al., 2012) was used to try and block the convergence of lytic granules to the MTOC from their dispersed localization in the cytoplasm. This would allow us to manipulate granule trafficking via blocking the motor protein function, instead of altering activating signal input. YTS, NK92 or eNK cells were treated with either ciliobrevin D or vehicle and conjugated with their respective target cells. Compared to vehicle control, ciliobrevin D treatment blocked lytic granule convergence in YTS (FIG. 6A, 6D), NK92 (FIG. 6B, E) and eNK (FIG. 6C, 6F) cells as determined by mean granule to MTOC distance. As a separate measure of granule dispersion, the standard deviation of the granule to MTOC distance was calculated to show the degree of scatter of individual lytic granules. This was also increased by ciliobrevin D in YTS (FIG. 6G), NK92 (FIG. 6H) and eNK (FIG. 6I) cells. CD107a staining was additionally performed to test whether ciliobrevin D treatment affected the exocytosis of lytic granules; the proportion of eNK cells that degranulated after treatment with ciliobrevin D increased (FIG. 13A), whereas in the NK cell lines it decreased (FIG. 13B-13C). In all cases, however, degranulation still occurred in the presence of ciliobrevin D.

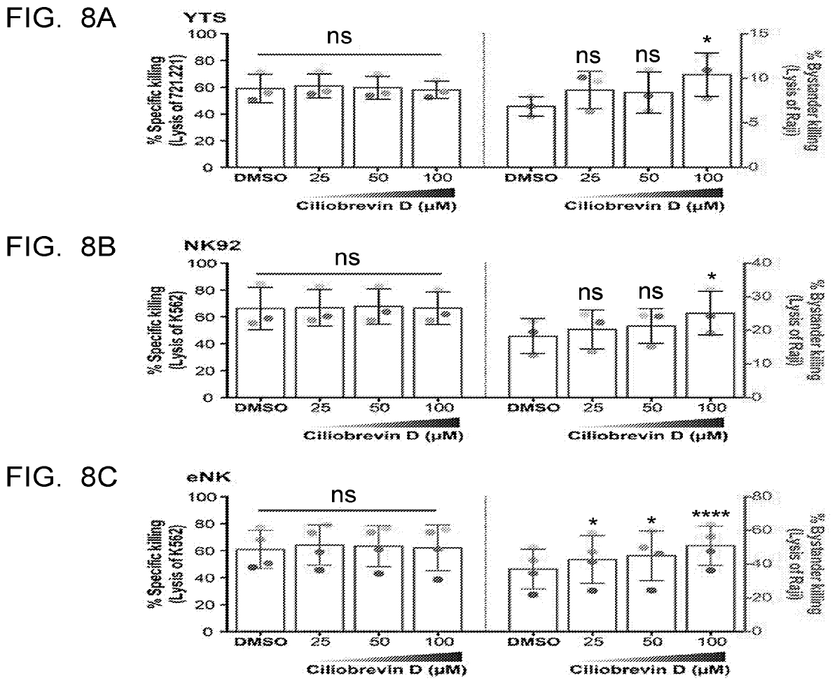

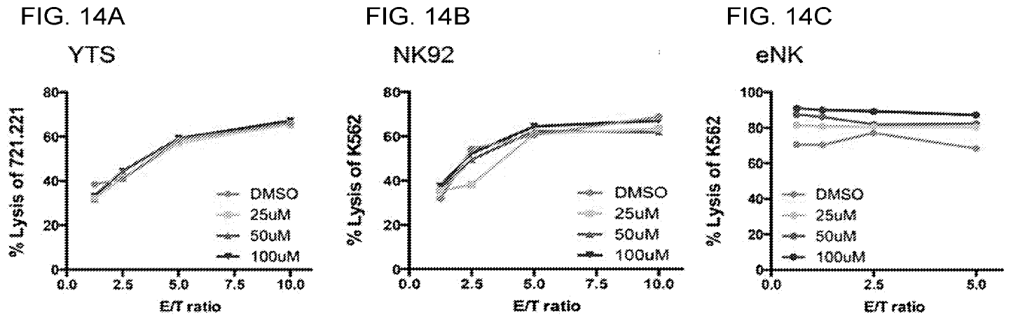

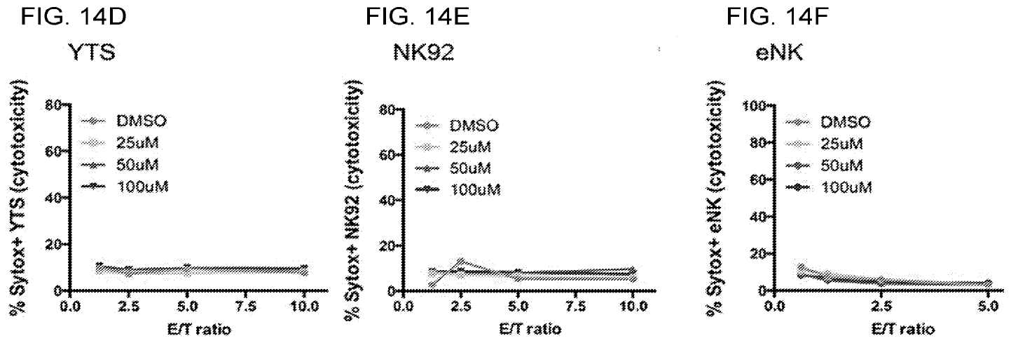

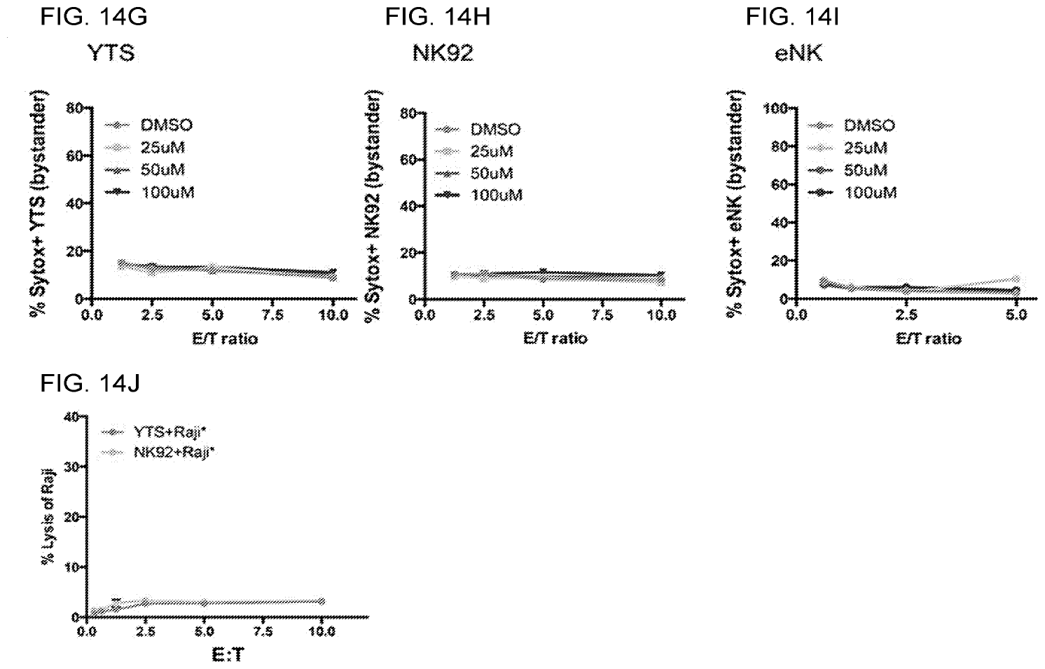

Because directed degranulation with converged granules promoted killing efficiency, ciliobrevin D was used to physically prevent convergence in a cell that had received a convergence signal. It was considered that this would allow us to abrogate the killing efficiency benefit imparted by lytic granule convergence. In order to measure killing of both the target and bystander cells in a single experiment, flow cytometry-based cytotoxicity assays were performed, wherein each cell line was labeled with a unique fluorescent dye in the presence of media containing SYTOX Blue viability indicator dye. NK cell-resistant B lymphoblastoid Raji cells were used as bystanders, and susceptible 721.221 or K562 were used as inciting target cells and both were incubated with YTS, NK92 or eNK cells that had been treated with DMSO or ciliobrevin D. In the absence of Raji bystander cells, ciliobrevin D did not affect killing of 721.221 or K562 cells (FIG. S5A-C). When Raji cells were added, ciliobrevin D-treated YTS (FIG. 8A), NK92 (FIG. 8B) and eNK (FIG. 8C) cells again showed no change in killing of 721.221 or K562 cells (FIG. 8A-C, left) but, now demonstrated killing of the bystander Raji cells in a dose-dependent manner (FIG. 8A-C, right). The viability of YTS (FIG. S5D, G), NK92 (FIG. 14E, 14H) and eNK (FIG. 14F, 14I) was not affected by DMSO or ciliobrevin D treatments. Using standard 51Cr-release assays, the inventors additionally analyzed the lysis of susceptible 721.221 and K562 cells by YTS (FIG. S4D) and NK92 (FIG. 13E) cells, respectively, with and without ciliobrevin D and found that ciliobrevin D did not substantively alter total killing. To ask if it promoted more collateral damage by NK cells 51Cr-labeled Raji cells were added (to serve as innocent bystanders) along with unlabeled 721.221 or K562 target cells. Compared to DMSO-treated control YTS (FIG. 13D) and NK92 (FIG. 14E) cells, those treated with ciliobrevin D caused more non-specific lysis of the Raji cells. YTS or NK92 cells co-cultured with 51Cr-labeled Raji cells in the absence of 721.221 or K562 were unable to kill Raji cells (FIG. 14J). Thus ciliobrevin D treatment prevented lytic granule convergence and promoted greater collateral damage even when a convergence signal was present. Convergence therefore serves to protect innocent bystander cells in complex cellular environments.

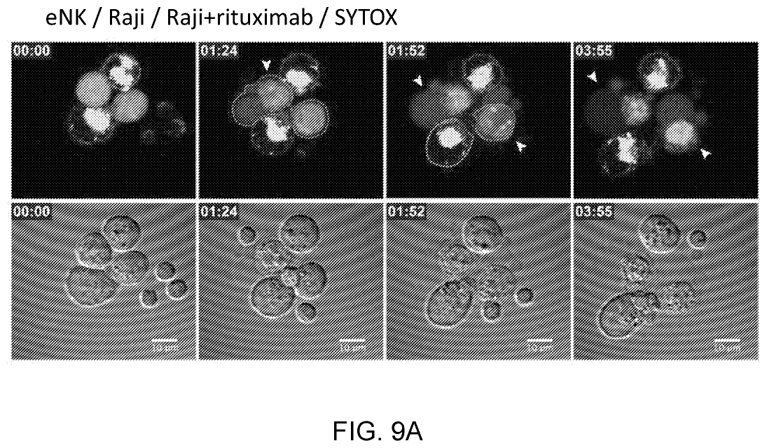

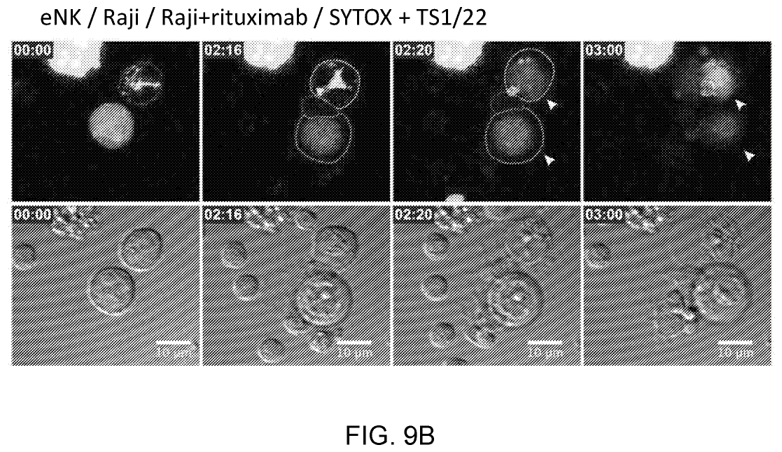

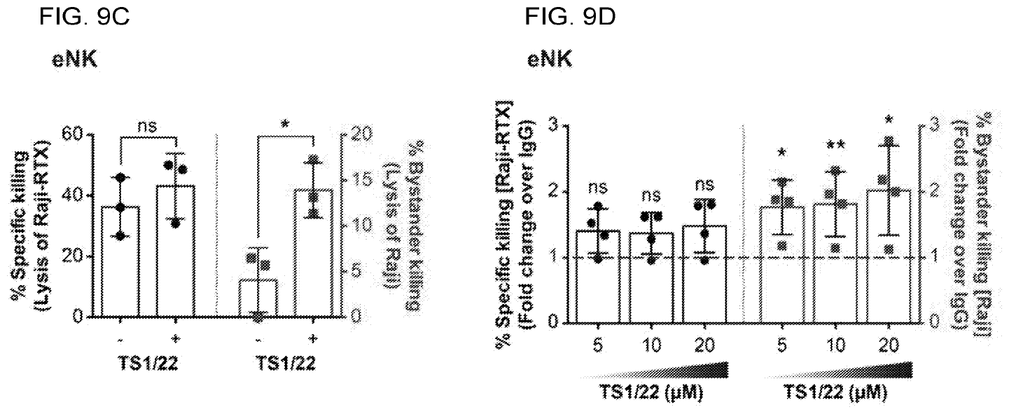

To apply this concept in the context of physiologic antibody-dependent cell mediated cytotoxicity (ADCC) a human therapeutic monoclonal antibody, Rituximab (RTX) was used along with NK cell resistant Raji cells that naturally express the antigen recognized by RTX. Here UGATm was utilized to aggregate eNK cells with yellow dye-labeled uncoated Raji cells and green dye-labeled RTX-coated Raji cells all in the presence of SYTOX Blue viability dye. In this setting there was preferential killing of the RTX-coated Raji cells (FIG. 9A, 9C). When LFA-1 engagement was prevented, however, by pre-treating eNK cells with LFA-1-blocking antibody there was significantly increased killing of uncoated Raji cells (FIG. 9B, 9C). The same result was found on a population basis when these same cells combinations were used in a 51Cr-release assay: the addition of LFA-1 blockade led to greater destruction of the otherwise resistant Raji cells (FIG. 9D). Thus the engagement of LFA-1 by the inciting target cell prevents bystander killing. This in concert with ciliobrevin-D findings uncovers a potentially useful strategy to control cytotoxic cells to promote collateral damage.

Significance of Certain Embodiments

NK cell activation triggers stepwise cellular events leading to secretion of lytic granules and lysis of diseased cells. A characteristic step in this process of unknown significance is that of lytic granule convergence to the MTOC after NK cell activation. As shown previously, lytic granule convergence precedes MTOC/granule polarization, is independent of actin rearrangement/microtubule dynamics and calcium mobilization, and depends on specific signaling requirements downstream of β2 integrin (James et al., 2013; Mentlik et al., 2010; Zhang et al., 2014). While the process of convergence itself is established, its contribution to cytotoxicity has not been. Aiming to elucidate how lytic granule convergence might contribute to NK cell cytotoxicity, one of our major challenges was to specifically access and block granule convergence without impairing the downstream cytolytic functions. To overcome this difficulty, a surrogate experimental target cell system employing Drosophila S2 cells was utilized. Advancing prior work in the signaling for convergence (James et al., 2013; Zhang et al., 2014), NK cells were provided with specific combinations of signal inputs to precisely experimentally control granule convergence and degranulation: LFA-1 ligation triggers lytic granule convergence but not degranulation, whereas CD16 ligation induces degranulation without convergence. This dichotomy was used to functionally dissect any contribution of convergence to cytotoxicity.

Through the use of an UGATm system the inventors were able to precisely govern and image the beginning of contact between NK and target cells to fully appreciate the positioning of lytic granules after reception of the activation signaling (while monitoring the viability of all cells). This approach allowed us to move beyond chance events and methodically collect replicates for quantitative analysis of granule dynamics. When combined with NK cells expressing our previously reported degranulation indicator (Rak et al., 2011) granule positioning was able to be coordinated with the directionality of secretion. The inventors also were able to complement these approaches with imaging flow cytometry for the purpose of mass data collection with spatial localization providing the additional advantage of not having to adhere cells to glass surfaces. Combining these approaches, it was demonstrated that granule convergence induced by LFA-1 engagement leads to more targeted secretion at the IS. In comparison, granules remaining dispersedly distributed in NK cells conjugated with S2-IgG cells degranulated not only at the IS but also at other locations (a result confirmed using anti-receptor antibody-coated polystyrene beads and LAMP1-pHluorin cells). This dichotomy allowed us to ask how directional versus non-directional degranulation impacts the cytolytic activity of NK cells. It was considered that convergence served as a preparatory step to help promote targeted killing of transformed target cells while preserving the healthy surrounding tissue.

Without converged granules, NK cell killing efficiency was drastically reduced when measured by specific lysis of inciting target cells over time. Since eNK cells conjugated with S2-IgG or S2-IC1-IgG cells resulted in comparable degranulation levels and similar IS geometries, the lack of converging granules is likely to be the major cause of the reduced killing efficiency in our study. Therefore, lytic granule convergence serves as a prerequisite step enabling NK cells to compress their cytolytic cargo and allow for focused release of their lytic contents to promote efficient target cell lysis. Thus when considering how an NK cell may navigate a complex tissue comprised mostly of healthy cells to “seek and destroy” diseased cells, their convergence strategy emerges as fundamental.

As an important demonstration, in our experiments CD16 signaling alone caused significantly higher non-specific killing of bystander target cells compared to the combined signals of CD16 and LFA-1. As previously shown, NK cells from leukocyte adhesion deficiency-1 patients, who lack LFA-1, could not converge lytic granules and had inefficient cytolytic activity (James et al., 2013). As ICAM-1 is expressed on a variety of mammalian cell types (Hubbard and Rothlein, 2000; Ramos et al., 2014) it likely serves as a near omnipresent facilitator to promote NK cell targeted attacks within healthy tissues as simulated using our UGATm-induced cell aggregates. Therefore, LFA-1 promoting lytic granule convergence may be evolutionarily preserved as the prerequisite mechanism to ensure the specificity of NK cell cytotoxicity. This concept is underscored further by our results in LFA-1 blockade as doing so in the presence of an inciting IgG-coated physiologic target cell causes the increased death of non-inciting surrounding cells.

It is relevant to note that while CD16 signaling alone from an opsonized target cell does not lead to granule convergence in NK cells, the combination of CD16 and LFA-1 in our experiments further reduced the average distance of lytic granules to the MTOC compared to LFA-1 alone. This suggests that CD16 signaling in concert with the true convergence signal can also add to lytic granule convergence. This may also in part explain why very high concentrations of anti-S2 rabbit antiserum had been previously observed induce low level of lytic granule convergence in eNK cells (Zhang et al., 2014). This may have additionally been a feature of that study having using diluted rabbit serum, as the inventors did not identify this effect in our experiments using purified anti-S2 IgG. Irrespective, the concepts do speak to the important overlap in and interplay between LFA-1 and CD16 signaling pathways, even though that of CD16 is missing key elements to efficiently initiate convergence (Zhang et al., 2014). In this light, the upstream signaling components common to both pathways remains to be fully established.

The preformed lytic granules in NK cells are lysosome-related organelles that comprise a cell type-specific subcellular compartment, which as the secretory lysosomes in haematopoietic cells are comparable to the Weibel-Palade bodies (WPB) in endothelial cells (Marks et al., 2013). Secretory lysosomes are dual-function organelles that share features with both conventional secretory and endocytic pathways (Blott and Griffiths, 2002). Examples include lytic granules in NK cells and CTLs, dense granules in platelets, histamine-containing granules in mast cells and basophils as well as melanosomes in melanocytes. Secretory lysosomes share certain molecular requirements for secretion. For example, Rab27a facilitates tethering of lytic granules (Elstak et al., 2011; Kurowska et al., 2012), melanosomes (Hume and Seabra, 2011) and WPB (Zografou et al., 2012); partially overlapping vSNARE and Munc complexes promote docking of lytic granules (Feldmann et al., 2003) and dense granules (Ren et al., 2010). As a distinct characteristic of NK cells and CTLs, however, lytic granules traffic to the MTOC using dynein (Mentlik et al., 2010; Ritter et al., 2015) prior to polarizing towards the cell periphery allowing for the tethering and docking processes. Melanocytes for example, also transport melanosomes in a minus-end-directed manner utilizing dynein function, but that only serves to aggregate melanosomes at the MTOC, which prevents secretion of pigments (Nascimento et al., 2003; Nilsson et al., 1996) and as such is more typical of secretory lysosome regulation across cell types. Of note, NK cells do converge lytic granule in non-cytolytic conjugates (Mentlik et al., 2010), possibly leading to this same outcome—to prevent undesired granule secretion onto innocent targets. Thus, when an NK cell enters a cellular tissue environment and is exposed to adhesion via an integrin signal, it likely first converges its granules to protect the tissue itself and provide the opportunity to further sense and integrate signals for targeted killing of the diseased cells within a mostly healthy tissue.

In light of the mechanism driving convergence, it was considered that through specifically inhibiting dynein in NK cells that have received a convergence activation signal, this protective mechanism can be bypassed—forcing lytic granules to degranulate non-directionally leading to increased non-discriminant bystander killing. As a proof of concept, ciliobrevin D, a small molecule inhibitor of cytoplasmic dynein, was used to block granule convergence in NK cells Indeed, ciliobrevin D-treatment led to a dose-dependent increase of collateral killing against the non-susceptible bystander cells while maintaining the killing rate of the sensitive target cells. This approach allowed us to demonstrate how an inability to converge lytic granules in activated NK cells may nonspecifically kill neighboring innocent cells in a more physiologically relevant setting. Along these lines, the inventors also approached the same concept using the blockade of LFA-1 to prevent convergence, but here with the clinically relevant IgG molecule RTX to provide the degranulation signal. As was the case with ciliobrevin D-treatment, blocking convergence by blocking LFA-1 promoted the killing of otherwise non-susceptible bystander cells.

Importantly, these observations provide a potential therapeutic insight and lead, as nondirectional degranulation may be useful in the setting of cellular therapies using cytotoxic cells or therapeutic monoclonal antibodies (like RTX). Cytotoxic cells infused into patients for anti-cancer therapy (also referred to as “cell therapy”) has led to major advances and provided great hope in clinical medicine (Fesnak et al., 2016; Jackson et al., 2016). Cell therapy cells are highly activated and express integrin as well as exogenously introduced activation receptors to allow them to destroy cancer cells after specific recognition of a cancer-specific ligand. Initial cellular studies have demonstrated converged and polarized lytic granules in therapy cells conjugated with tumor cell targets (Hegde et al., 2016). While cytotoxic cells excel in eliminating individual diseased cells, an established tumor contains many diseased cells some of which are in the process of immune escape. Thus converged and polarized lytic granules in therapy cells may not be the most effective in the eradication of a massive tumor by those therapy cells that have trafficked into the tumor environment. In this instance preventing lytic granule convergence in therapy cells (for example, by pre-treating them with ciliobrevin-D itself or anti-LFA-1) prior to their infusion into a patient may allow for broad scale diffuse degranulation in, with “collateral damage” mediated by transferred cells that have received a degranulation signal. This could allow therapy cells that have entered a tumor to promote more widespread killing potentially providing greater tumor cell killing per therapy cell as well as promoting killing of tumor cells in the process of immune escape. This is likely a different task than what NK cells have evolved to excel at, which is precision seek and destroy missions protecting healthy surrounding tissues and the killing of single diseased cells before they establish a tumor. The same approach could be envisioned as an adjunct therapy for patients receiving a monoclonal antibody to treat cancer—although this would require an additional treatment to patients as the therapeutic antibodies utilize a patient's endogenous cytotoxic cells. Thus, while effective, there may be substantive room for improvement in these immune-based therapies by exploiting the cell biology of cytotoxicity and lytic granule positioning.

Taken together, the inventors have shown for the first time that NK cells can cause collateral damage to healthy bystander cells using non-directed degranulation. That is, lytic granule convergence plays an essential role in regulating the pointed and focused secretion of the cytolytic granule contents. As such lytic granule convergence in NK cells and CTLs, serves as the primary regulatory step for promoting precision of targeted killing and diminishing occurrence of bystander killing, in specific embodiments. The nondirectional degranulation, pathway induced by CD16 signaling however is likely to also exist for evolutionarily relevant reasons. For one, it may serve as a natural “last chance” effort against growing tumors as ICAM-1 expression is often downregulated in established tumor cells which renders them resistant to CTL lysis (Blank et al., 2005; Hamai et al., 2008), but may allow for an NK cell perceived danger signal to provide diffuse degranulation in the tumor cell environment (Gras Navarro et al., 2015; Stojanovic and Cerwenka, 2011). More importantly however, non-directed secretion may function in the elimination of opsonized non-human pathogens in a pathogen rich environment (Jones et al., 2009; Lu et al., 2014) allowing an entering NK cell to destroy large numbers of organisms, only a few of which may be coated with host-produced IgG (directly analogous to the S2-IgG cells). Thus, convergence plays a role in NK cell cytotoxicity and that can exploited for tailoring and improving immunological therapies.

Materials and Methods

Cell Lines and Ex Vivo NK Cells—

The immortalized human NK cell lines YTS and NK92 were maintained as described previously (James et al., 2013). The YTS-CD16 cell line was a kind gift from Dr. John Coligan and Dr. Konrad Krzewski (Peruzzi et al., 2013). The NK92-CD16 cell line was a kind gift from Dr. Kerry Campbell (Binyamin et al., 2008). Human ex vivo NK (eNK) cells were prepared from peripheral blood of healthy donors by standard Ficoll-Paque isolation followed by negative selection (Miltenyi Biotec). All human blood samples were obtained in accordance with the Institutional Review Board for the Protection of Human Subjects of Baylor College of Medicine. The Drosophila S2 and S2-ICAM-1 (S2-IC1) Schneider cell line were kindly provided by Dr. Dongfang Liu and were used as surrogate targets for YTS-CD16, NK92-CD16 and eNK cells, which were maintained as previously described (James et al., 2013). The 721.221 cell line was used as susceptible target cells for YTS and YTS-CD16 cells and K562 cell line as target cells for NK92, NK92-CD16 cells and eNK cells. The Raji cell line was used as non-susceptible target cells in the bystander cytotoxicity assay for YTS, NK92 and eNK cells. YTS-CD16 cells were transduced to stably express LAMP-1-pHluorin as described previously (Rak et al., 2011).

Live Cell Confocal Microscopy—

YTS-CD16 and eNK cells were incubated with 5 μM LysoTracker Red DND-99 (Thermo Fisher) for 30 min at 37° C. and washed. S2 and S2-IC1 cells were pre-incubated with anti-S2 polyclonal IgG for 30 min, washed, and labeled with cell proliferation dye eFluor670 (eBioscience). For bystander killing assays, S2 cells were labeled with CellTrace CFSE cell proliferation dye (Thermo Fisher) to differentiate them from the S2-IgG or S2-IC1-IgG cells. NK and target cells were mixed at a ratio of 1:3 to a final volume of 200 μl complete R10 medium supplemented with 5 μM SYTOX Blue viability dye (Thermo Fisher). Cell mixtures were then added directly onto the chip of the UGATm device to allow clustering of cells in the central area of each microwell (Christakou et al., 2013). Cells in the microwells were imaged using a Leica SP8 laser scanning confocal microscope with a 100× magnification 1.4 numerical aperture objective. Excitation was provided by a UV laser at 405 nm and tunable white light laser at 488, 561, and 647 nm. Emission was detected with HyD detectors, and images were collected in a single z-plane at one frame every 4 or 30 min for 120 min. Data were acquired with LAS AF software (Leica) and subsequently exported to Volocity software (PerkinElmer) for further analysis. For imaging of ADCC, eNK cells, Raji cells and RTX-coated Raji cells were labeled with LysoTracker Red DND-99, cell proliferation dye eFluor670 and CellTracker Green (Thermo Fisher), respectively. Cells were then mixed at a 1:3:3 ratio in 200 μl R10 medium supplemented with SYTOX blue viability dye in the presence of control murine IgG1 mAB or anti-CD11a blocking mAb. Images were taken every 4 min for 4 hours as described above. For live degranulation imaging, YTS-CD16-LAMP1-pHluorin cells were loaded with LysoTracker Red and incubated with S2-IgG or S2-IC1-IgG cells labeled with eFluor670 proliferation dye, or antibodycoated polystyrene beads. 10 μm polystyrene beads (Polysciences) were coated with 10 μg of anti-CD16 mAb (clone 3G8), or anti-CD16 and anti-CD8 (clone B34) mAb for 30 min at 37° C., washed once with PBS and added to the YTS-CD16-LAMP1-pHluorin cells. Cell mixtures were then immediately applied onto the microwells for imaging as described previously (Rak et al., 2011).

Fixed Cell Confocal Microscopy—

YTS-CD16, NK92-CD16 or eNK cells co-cultured with S2, S2-IgG, S2-IC1 or S2-IC1-IgG cells for 15 min were adhered to poly-L-lysine-coated glass slides for 20 min at 37° C. The surrogate target S2 cells were previously labeled with CellTracker Orange (Thermo Fisher). Fixation, permeabilization and staining were performed as described (Banerjee et al., 2007). The reagents/antibodies were used as the following sequence for optimized results: 1) biotinylated monoclonal mouse-anti-tubulin (Invitrogen); 2) Streptavidin-Pacific Blue (Invitrogen); 3) FITC-conjugated mouse-anti-perforin clone δG9 (BD). Slides were mounted with 0.15 mm coverslips (VWR Scientific) using ProLong AntiFade (Invitrogen). The image acquisition settings were as described in live cell confocal microscopy and all transmitted light images were specifically from the focal plane of the MTOC of the NK cell which may not have been ideal for those images, especially the conjugated target cell, and are thus provided for orientation only.

Flow Cytometry-Based Conjugation Assay—

YTS-CD16, NK92-CD16 and eNK cells were labeled with CellTrace CFSE for 10 min at room temperature. S2 and S2-IC1 cells were pre-incubated with S2 antiserum or anti-S2 polyclonal IgG antibody at a final dilution of 1:1000 for 30 min at room temperature and washed three times. S2, S2-IgG, S2-IC1 and S2-IC1-IgG cells were then labeled with cell proliferation dye eFluor 670 for 5 min at room temperature. 105 NK cells and 2×105 target cells were mixed in 200 μl complete R10 and incubated for the indicated times at 37° C. (0, 10, 30 or 60 min), vortexed, and fixed with 1% paraformaldehyde in PBS. Cell mixtures were run on an LSRFortessa flow cytometer (Becton Dickinson) and data analyzed using FlowJo X (TreeStar, Inc).

Flow Cytometry-Based Degranulation Assay—

YTS-CD16, NK92-CD16 and eNK cells were labeled with CellTrace CFSE for 10 min at room temperature. S2 and S2-IC1 cells were pre-incubated with anti-S2 polyclonal IgG antibody at a final dilution of 1:1000 for 30 min at room temperature and washed three times. S2, S2-IgG, S2-IC1 and S2-IC1-IgG cells were then labeled with cell proliferation dye eFluor 670 for 5 min at room temperature. 105 NK cells and 2×105 target cells were mixed in 200 μl complete R10 and incubated for 2 hours at 37° C. in the presence of anti-CD107a antibody and GolgiStop (BD), fixed with 1% paraformaldehyde in PBS and analyzed as described in flow-based conjugation assay.

Flow Cytometry-Based Cytotoxicity Assay—

YTS-CD16, NK92-CD16 and eNK cells were labeled with CellTrace CFSE for 10 min at room temperature, washed, and treated with ciliobrevin D or DMSO control as described below in inhibitors. 721.221 and K562 cells were labeled with cell proliferation dye eFluor 670 for 5 min at room temperature. Raji cells were labeled with PKH-26 according to manufacture's protocol. NK cells were then mixed with the corresponding targets and Raji cells at a final volume of 200 μl complete R10 and incubated at 37° C. for 2 hr. Cells were then labeled with SYTOX Blue viability dye for 5 min at room temperature, washed, and run on LSRFortessa flow cytometer (BD).

⁵¹Cr-Release Assay—

Standard 51Cr-release assays were performed as previously described (Orange et al., 2011). Where indicated, 51Cr51-labeled target or “bystander” cells were mixed with unlabeled target or “bystander” cells to test the indirect lysis of targets. For ADCC, Raji cells were incubated with 20 μg/ml of rituximab in complete R10 medium for 20 min at 37° C., and washed three times before adding to eNK cells.

Imaging Flow Cytometry—

YTS-CD16, NK92-CD16 and eNK cells were labeled with CellTrace CFSE. S2-IgG and S2-IC1-IgG cells were labeled with cell proliferation dye eFluor 670. NK cells and target cells were mixed at 1:2 ratio in 200 μl complete R10 supplemented with anti-CD107a antibody and BD GolgiStop™, incubated for 30 min at 37° C., vortexed, and fixed with 1% paraformaldehyde in PBS. Cell mixtures were run on Amnis MKII imaging flow cytometer (EMD Millipore) and data were analyzed using IDEAS software (Viswanath et al., 2016).

Anti-S2 Polyclonal Antibody—

Whole S2 cell pellet was used for immunization of rabbits. Rabbit serum was collected after high dose extension and purified using protein A (Thermo Fisher Custom antibody production).

Inhibitors—

Where applicable, YTS, NK92 and eNK cells were pretreated with 25, 50 or 100 mM ciliobrevin D (Millipore) for 30 min then washed. DMSO was used as the vehicle control for all inhibitor experiments. The inhibitor was also added into the co-culture with target cells.

LFA-1-Blocking Monoclonal Antibody—

For live cell confocal microscopy, eNK cells were pretreated with 20 μg of anti-CD11a LFA-1 blocking mAb (clone TS1/22) or murine IgG1 mAb (clone MOPC21) for 15 min at 37° C. before incubation with the dye-labeled target cells. For standard four-hour 51Cr-cytotoxicity assay, eNK cells were pretreated with IgG, or 5, 10, or 20 μg of LFA-1-blocking mAb before incubation with the target cells.

Analysis of Synapse Geometry—

Raw image sequences of YTS-CD16 co-cultured with S2 cells expressing ICAM-1 and IgG (S2-IC1-IgG), or IgG (S2-IgG) or parental (S2) were used to characterize the general geometry of the synapse formed between effector and target. Briefly, the transmitted light channel was contrasted and smoothed using Gaussian blur tool with a sigma radius of 1 pixel to facilitate the manual outlining of the contours of the cells in ImageJ (v 1.51f). The perimeter of the cells was measured along with the proportion of their respective outline involved in the IS (area of overlapping perimeters between target and effector cells). The total amount of lytic granules in the effector cell was measured as the sum of intensity of the LysoTracker Red signal contained within the cell outline in all the focal planes. An axis perpendicular to the synapse was drawn in the effector cell and the length between the synapse and the opposite side of the cell was measured. The third of the length of this axis, closest from the synapse, was set as the limit of the pool of granules considered as polarized to the synapse. The proportion of the granule at the synapse was then expressed as a ratio of the total signal measured in this area divided by the total amount measured in the cell; both within the limits of the cell outline and in all focal planes acquired.

Image Analysis—

Raw image sequences were analyzed using Volocity software. For lytic granules, the centroid of individual lytic granule was designated automatically based on the fluorescence intensity of the corresponding staining (LysoTracker Red or perforin) (Mentlik et al., 2010). For MTOC, in fixed-cell confocal experiments, the point with the highest fluorescence intensity of α-tubulin staining was denoted as the localization of MTOC; in live-cell experiments, the geometric centroid of all the lytic granules in the cell was used to provide a measure of proximity of lytic granule regions. Lytic granule convergence to the MTOC or to the centroid of all granules was measured as previously described (Mentlik et al., 2010). Presentation images were contrast enhanced uniformly and in general were representative of near mean data from experimental repetitions.

Figure Preparation—

Fixed-cell confocal images and live-cell image sequences were post-processed in Image J. Contrast was adjusted using an empirically determined linear transformation and color-merged for display in the figures and supplemental movies. Where applicable, transmitted light and/or fluorescent channels were smoothed using Gaussian blur tool with a sigma radius of 1 pixel for display purposes. All quantitative analyses were performed on the raw data. For live degranulation images, maximum projection of all z-slices in the LysoTracker Red and pHluorin channels was used and only one z-slice for the transmitted light channel was used in the display.

Statistical Analysis—

Minimum number of samples required was determined using sample size calculations based upon preliminary data with a and β error levels of 1%. Means of three or more groups were compared using one-way ANOVA, and if significant difference was achieved, individual means were then compared using Student's t test. Error bars show ±SD. Differences were considered significant when p≤0.05 (*, p<0.05; **, p<0.01; ***, p<0.001; ****, p<0.0001).

### Example 2

### Controlling Lytic Granule Positioning to Harness Cytotoxicity in Therapy Car-Bearing Cells

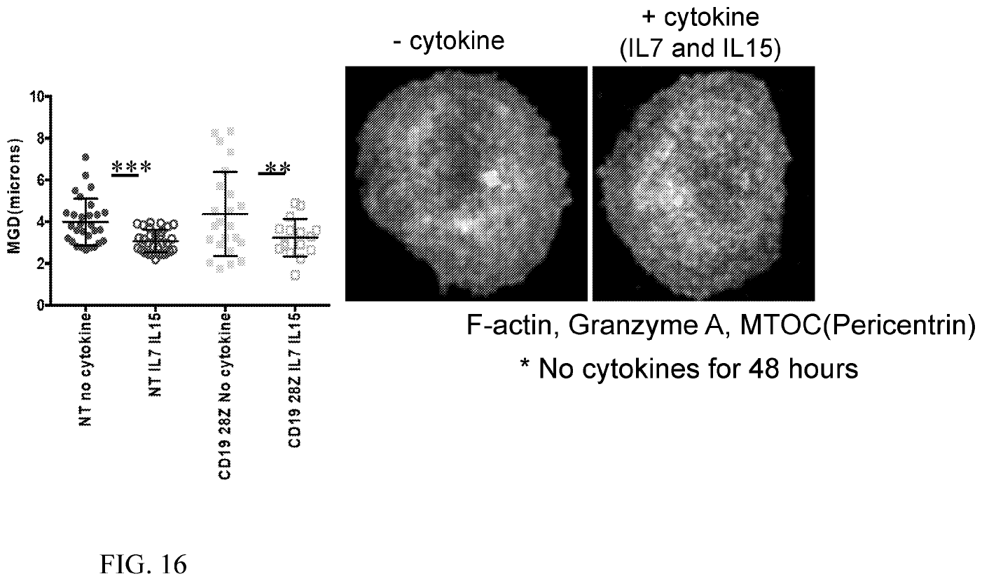

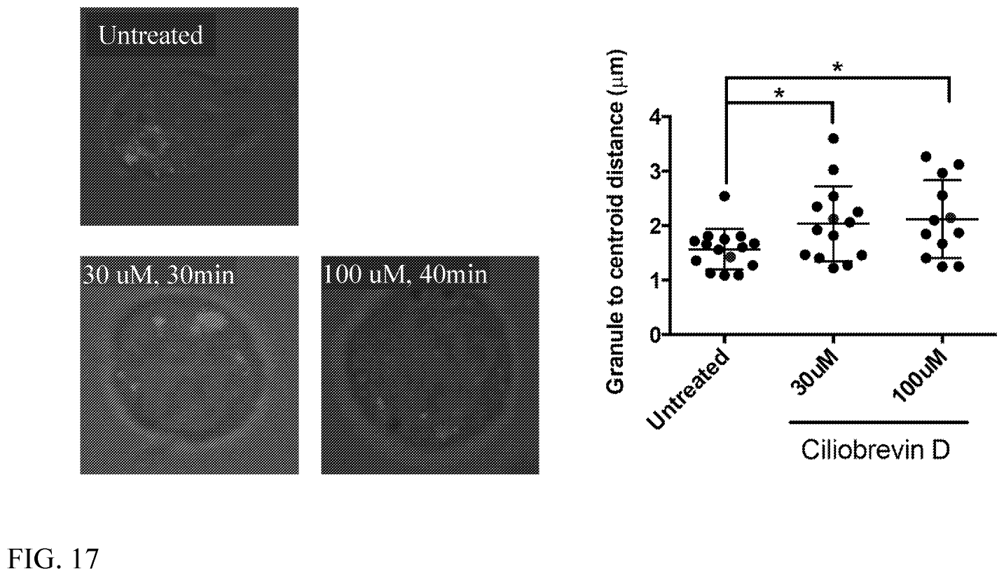

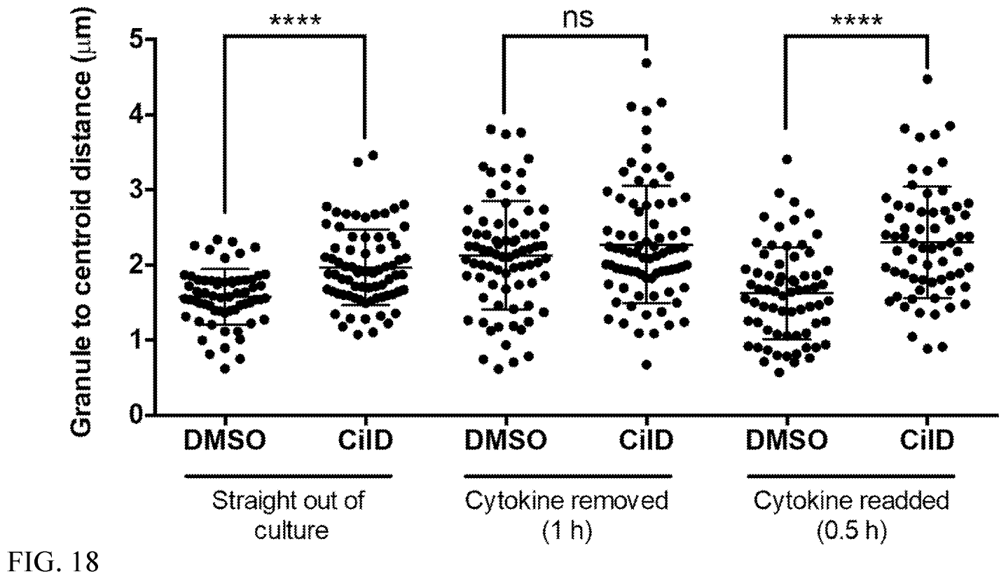

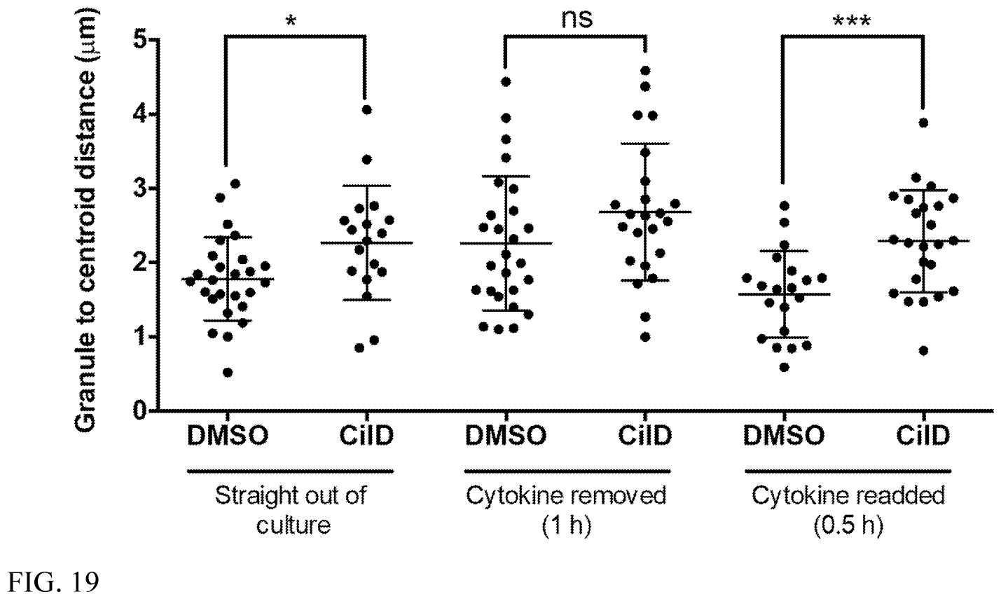

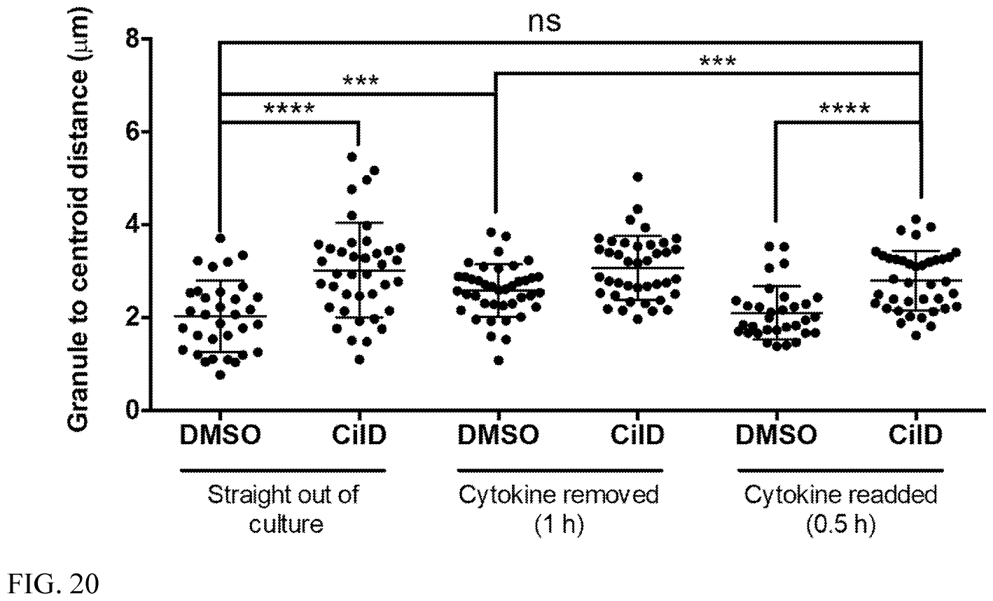

The inventors have also demonstrated convergence in multiple types of CAR-bearing cells, such as T cells, NK cells, and NK T cells. Specifically, the inventors have shown convergence in CD19 CAR T cells, CD19 CAR NK cells, PSCA CAR T cells, GD2 CAR NKT cells, HER2 CART cells, and glypican CAR T cells (FIG. 15A). Using a measurement of mean granule distance from the MTOC, CAR cells have varying degrees of converged lytic granules (FIG. 15B). FIG. 16 shows that when CAR-expressing cells are cultured in the presence of cytokines, the granules converge. However, ciliobrevin D can disperse lytic granules in CAR-bearing cells (FIG. 17) and blocks stimulation-induced convergence in CD19 CAR T cells (FIG. 18), CD19 CAR NK cells (FIG. 19) and GD2 CAR T cells (FIG. 20). This demonstrates that the principles of convergence initially described in human NK cells are applicable to engineered CAR-bearing cells. Furthermore, the use of ciliobrevin D to disperse granules in CAR-bearing cells indicates that the same approaches can be used to manipulate granule positioning in CAR-bearing cells as in ex vivo cells and cell lines.

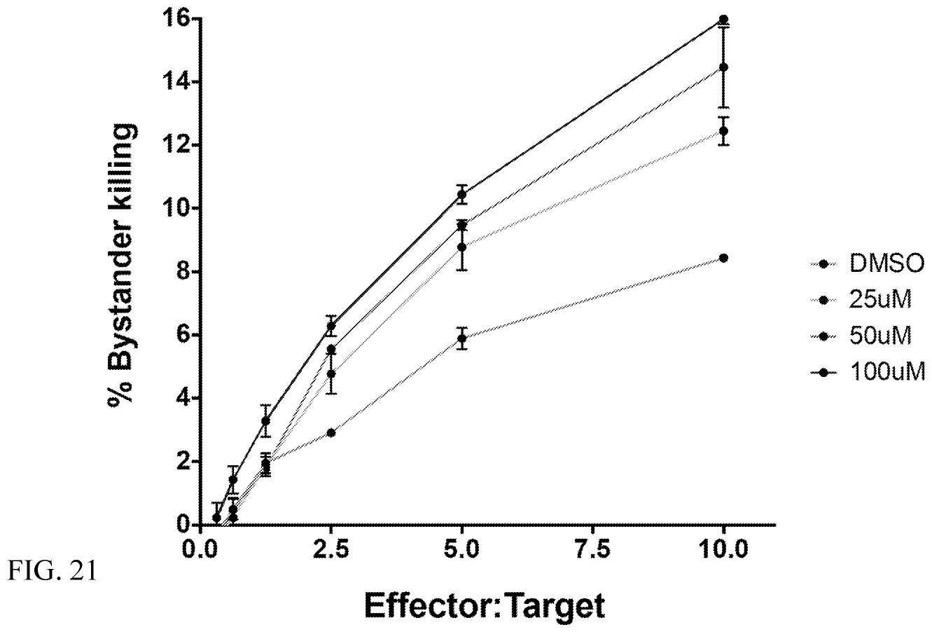

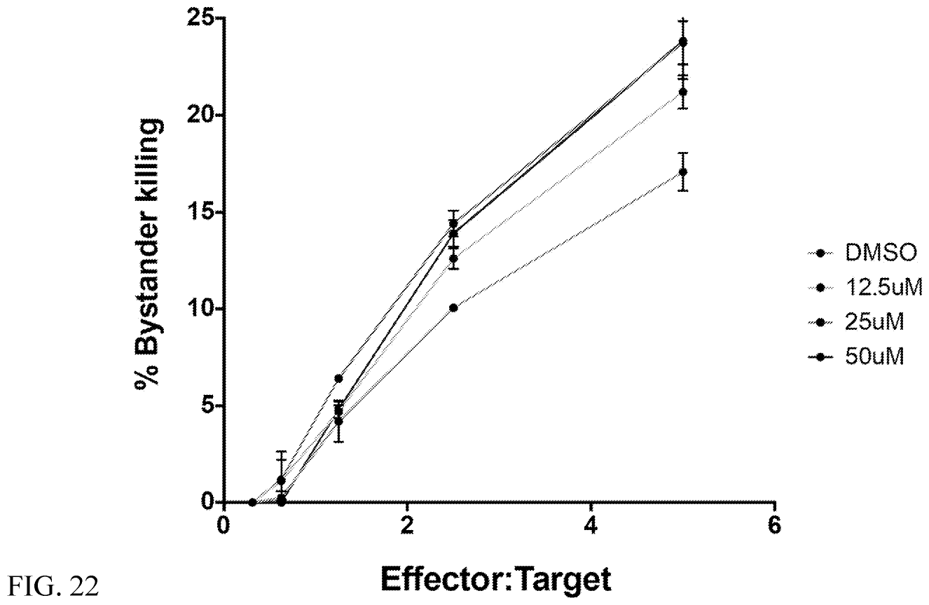

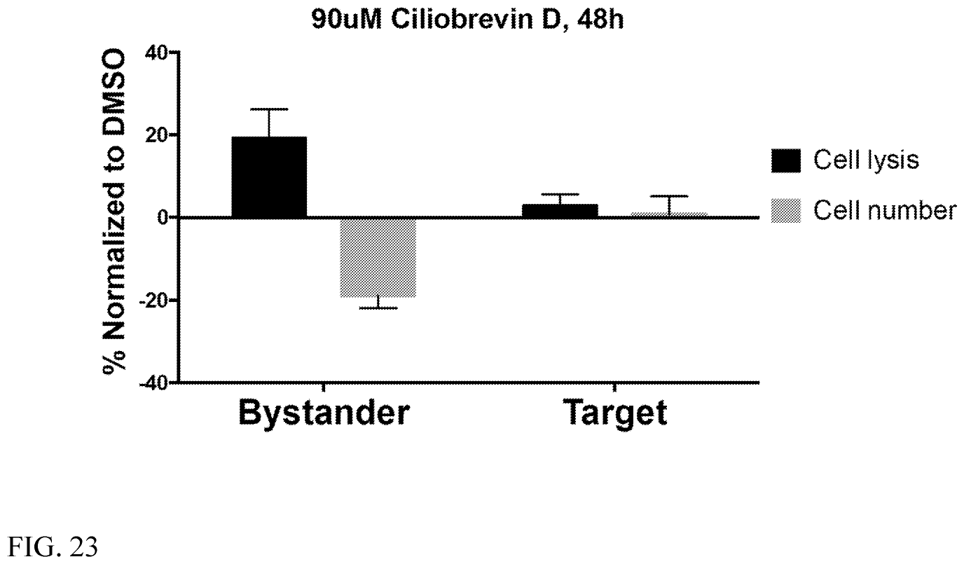

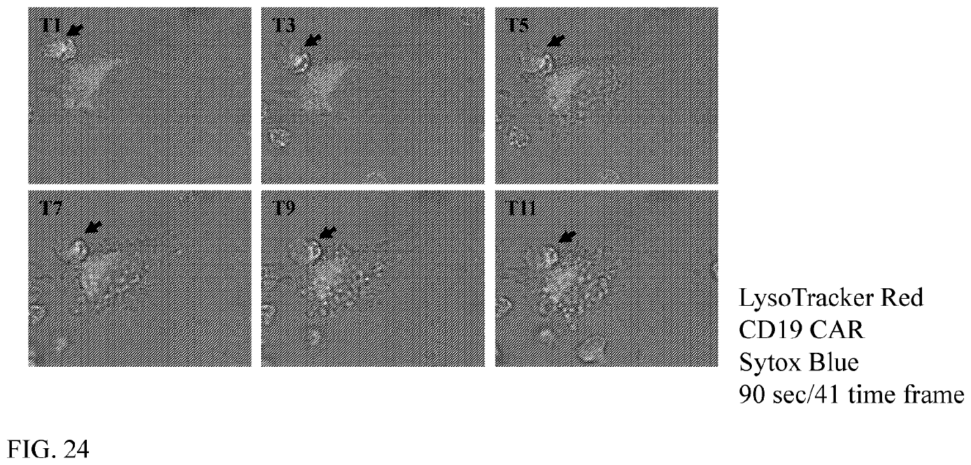

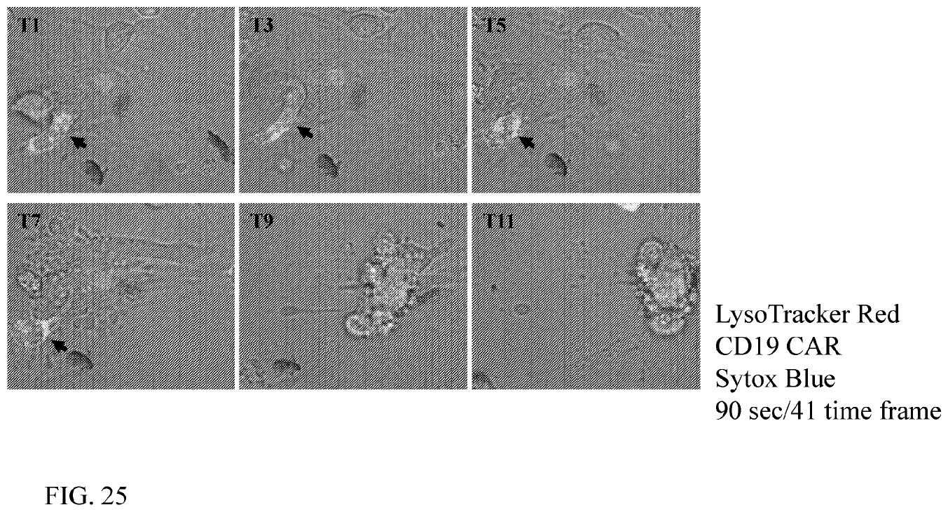

To demonstrate the functional utility of inhibitors of convergence as a modulator of CAR-bearing effector cells, the inventors showed that ciliobrevin D can also promote increased bystander killing from a variety of therapy CAR-bearing cells. In FIG. 21, results from a representative donor are demonstrated that showed dose-dependent increases of bystander killing with ciliobrevin D. Specifically, at 10:1 effector to target ratio, there are approximately 48%, 71% and 90% increase of bystander killing by CD19 CAR T cells treated with 25, 50 and 100 μM of ciliobrevin D, respectively, compared to DMSO control. Similarly, compared to DMSO, ciliobrevin D-treated CD19 CAR NK cells lyse approximately 24%, 39% and 40% more bystander cells at the concentration of 12.5, 25 and 50 μM, respectively (FIG. 22). Furthermore, GD2 CAR T cells treated with ciliobrevin D demonstrate ˜20% higher bystander killing while specific lysis remains similar to the DMSO control. To be noted, after 48-hour incubation, the total number of bystander cells decreases by ˜20%, whereas that of the target cells does not change. This indicates that blocking lytic granule convergence allows the therapy cells to increase killing of lysis-resistant bystander cells while maintaining lysis of susceptible target cells. FIG. 23 and FIG. 24 are time-lapse images of videos of control (FIG. 22) and ciliobrevin D-treated (FIG. 23) CD19 CAR T cells. The ciliobrevin D-treated CD19 CAR T cell shows dispersedly localized lytic granules throughout the video, from effector-target contact formation to specific lysis of the target cell (FIG. 23). In the control group, however, lytic granules converge immediately upon interacting with the target cell and remain tightly clustered until target is killed by the CD19 CAR T cell as indicated by uptake of the SYTOX Blue viability dye.

Therefore, CAR-bearing cells have pre-converged lytic granules, and standard immune cell culture conditions can stimulate convergence. CAR-bearing cells in conjugates with tumors have converged granules, and the CAR-bearing cell granules can be dispersed through dynein inhibition. The present disclosure provides a novel means of using dispersion of lytic granules as a means to re-route the evolutionary purpose of cytotoxic cells to re-program it into a therapy cell. This dispersion will impact cellular therapeutics in a variety of ways, including to address the inability to destroy tumor cells escaping detection by specific antigen targeting systems. Modulating dispersion will increase the capacity of a single therapy cell to specifically target a large number of tumor cells. Dispersion will provide an enhanced impact on immunosuppressive tumor microenvironments, where it is advantageous to kill bystander cells quickly. It will also reduce the number of cells needed for an effective cell therapy, thereby permitting a more tolerable dose of cells. As a major challenge within the field is generating enough therapy cells to locally target a tumor, dispersion will reduce the requirement for therapy cells to locally proliferate in order to overcome dense, tumor environments. For proliferation, dispersion addresses the requirement for proliferation of therapy cells to enable tumor clearance.

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.
Banerjee, P. P., R. Pandey, R. Zheng, M. M. Suhoski, L. Monaco-Shawver, and J. S. Orange. 2007. Cdc42-interacting protein-4 functionally links actin and microtubule networks at the cytolytic NK cell immunological synapse. The Journal of experimental medicine. 204:2305-2320.
Binyamin, L., R. K. Alpaugh, T. L. Hughes, C. T. Lutz, K. S. Campbell, and L. M. Weiner. 2008. Blocking NK cell inhibitory self-recognition promotes antibody-dependent cellular cytotoxicity in a model of anti-lymphoma therapy. Journal of immunology. 180:6392-6401.
Blank, C., I. Brown, A. K. Kacha, M. A. Markiewicz, and T. F. Gajewski. 2005. ICAM-1 contributes to but is not essential for tumor antigen cross-priming and CDS+ T cell-mediated tumor rejection in vivo. Journal of immunology. 174:3416-3420.
Blott, E. J., and G. M. Griffiths. 2002. Secretory lysosomes. Nature reviews. Molecular cell biology. 3:122-131.
Bryceson, Y. T., M. E. March, D. F. Barber, H. G. Ljunggren, and E. O. Long. 2005. Cytolytic granule polarization and degranulation controlled by different receptors in resting NK cells. The Journal of experimental medicine. 202:1001-1012.
Christakou, A. E., M. Ohlin, B. Vanherberghen, M. A. Khorshidi, N. Kadri, T. Frisk, M. Wiklund, and B. Onfelt. 2013. Live cell imaging in a micro-array of acoustic traps facilitates quantification of natural killer cell heterogeneity. Integrative biology: quantitative biosciences from nano to macro. 5:712-719.
Elstak, E. D., M. Neeft, N. T. Nehme, J. Voortman, M. Cheung, M. Goodarzifard, H. C. Gerritsen, P. M. van Bergen En Henegouwen, I. Callebaut, G. de Saint Basile, and P. van der Sluijs. 2011. The munc13-4-rab27 complex is specifically required for tethering secretory lysosomes at the plasma membrane. Blood. 118:1570-1578.
Eriksson, M., G. Leitz, E. Fallman, O. Axner, J. C. Ryan, M. C. Nakamura, and C. L. Sentman. 1999. Inhibitory receptors alter natural killer cell interactions with target cells yet allow simultaneous killing of susceptible targets. The Journal of experimental medicine. 190:1005-1012.
Feldmann, J I. Callebaut, G. Raposo, S. Certain, D. Bacq, C. Dumont, N. Lambert, M. Ouachee-Chardin, G. Chedeville, H. Tamary, V. Minard-Colin, E. Vilmer, S. Blanche, F. Le Deist, A. Fischer, and G. de Saint Basile. 2003. Munc13-4 is essential for cytolytic granules fusion and is mutated in a form of familial hemophagocytic lymphohistiocytosis (FHL3). Cell. 115:461-473.
Fesnak, A. D., C. H. June, and B. L. Levine. 2016. Engineered T cells: the promise and challenges of cancer immunotherapy. Nature reviews. Cancer. 16:566-581.
Firestone, A. J., J. S. Weinger, M. Maldonado, K. Barlan, L. D. Langston, M. O'Donnell, V. I. Gelfand, T. M. Kapoor, and J. K. Chen. 2012. Small-molecule inhibitors of the AAA+ ATPase motor cytoplasmic dynein. Nature. 484:125-129.
Gras Navarro, A., A. T. Bjorklund, and M. Chekenya. 2015. Therapeutic potential and challenges of natural killer cells in treatment of solid tumors. Frontiers in immunology. 6:202.
Ham, H., W. Huynh, R. A. Schoon, R. D. Vale, and D. D. Billadeau. 2015. HkRP3 is a microtubule-binding protein regulating lytic granule clustering and NK cell killing. Journal of immunology. 194:3984-3996.
Hamai, A., F. Meslin, H. Benlalam, A. Jalil, M. Mehrpour, F. Faure, Y. Lecluse, P. Vielh, M. F. Avril, C. Robert, and S. Chouaib. 2008. ICAM-1 has a critical role in the regulation of metastatic melanoma tumor susceptibility to CTL lysis by interfering with PI3K/AKT pathway. Cancer research. 68:9854-9864.
Hegde, M., M. Mukherjee, Z. Grada, A. Pignata, D. Landi, S. A. Navai, A. Wakefield, K. Fousek, K. Bielamowicz, K. K. Chow, V. S. Brawley, T. T. Byrd, S. Krebs, S. Gottschalk, W. S. Wels, M. L. Baker, G. Dotti, M. Mamonkin, M. K. Brenner, J. S. Orange, and N. Ahmed. 2016. Tandem CAR T cells targeting HER2 and IL13Ralpha2 mitigate tumor antigen escape. The Journal of clinical investigation. 126:3036-3052.
Hubbard, A. K., and R. Rothlein. 2000. Intercellular adhesion molecule-1 (ICAM-1) expression and cell signaling cascades. Free radical biology & medicine. 28:1379-1386.
Hume, A. N., and M. C. Seabra. 2011. Melanosomes on the move: a model to understand organelle dynamics. Biochemical Society transactions. 39:1191-1196.
Jackson, H. J., S. Rafiq, and R. J. Brentjens. 2016. Driving CAR T-cells forward. Nature reviews. Clinical oncology. 13:370-383.
James, A. M., H. T. Hsu, P. Dongre, G. Uzel, E. M. Mace, P. P. Banerjee, and J. S. Orange. 2013. Rapid activation receptor- or IL-2-induced lytic granule convergence in human natural killer cells requires Src, but not downstream signaling. Blood. 121:2627-2637.
Jones, G. J., J. C. Wiseman, K. J. Marr, S. Wei, J. Y. Djeu, and C. H. Mody. 2009. In contrast to antitumor activity, YT cell and primary NK cell cytotoxicity for Cryptococcus neoformans bypasses LFA-1. International immunology. 21:423-432.
Katz, P., A. M. Zaytoun, and J. H. Lee, Jr. 1982. Mechanisms of human cell-mediated cytotoxicity. III. Dependence of natural killing on microtubule and microfilament integrity. Journal of immunology. 129:2816-2825.
Kurowska, M., N. Goudin, N. T. Nehme, M. Court, J. Garin, A. Fischer, G. de Saint Basile, and G. Menasche. 2012. Terminal transport of lytic granules to the immune synapse is mediated by the kinesin-1/S1p3/Rab27a complex. Blood. 119:3879-3889.
Laan, L., N. Pavin, J. Husson, G. Romet-Lemonne, M. van Duijn, M. P. Lopez, R. D. Vale, F. Julicher, S. L. Reck-Peterson, and M. Dogterom. 2012. Cortical dynein controls microtubule dynamics to generate pulling forces that position microtubule asters. Cell. 148:502-514.
Lanier, L. L. 2005. NK cell recognition. Annual review of immunology. 23:225-274.
Liu, D., J. A. Martina, X. S. Wu, J. A. Hammer, 3rd, and E. O. Long. 2011. Two modes of lytic granule fusion during degranulation by natural killer cells. Immunology and cell biology. 89:728-738.
Lu, C. C., T. S. Wu, Y. J. Hsu, C. J. Chang, C. S. Lin, J. H. Chia, T. L. Wu, T. T. Huang, J. Martel, D. M. Ojcius, J. D. Young, and H. C. Lai. 2014. NK cells kill mycobacteria directly by releasing perforin and granulysin. Journal of leukocyte biology. 96:1119-1129.
Mace, E. M., P. Dongre, H. T. Hsu, P. Sinha, A. M. James, S. S. Mann, L. R. Forbes, L. B. Watkin, and J. S. Orange. 2014. Cell biological steps and checkpoints in accessing NK cell cytotoxicity. Immunology and cell biology. 92:245-255.
March, M. E., C. C. Gross, and E. O. Long. 2010. Use of transfected Drosophila S2 cells to study NK cell activation. Methods in molecular biology. 612:67-88.
Marks, M. S., H. F. Heijnen, and G. Raposo. 2013. Lysosome-related organelles: unusual compartments become mainstream. Current opinion in cell biology. 25:495-505.
Mentlik, A. N., K. B. Sanborn, E. L. Holzbaur, and J. S. Orange. 2010. Rapid lytic granule convergence to the MTOC in natural killer cells is dependent on dynein but not cytolytic commitment. Molecular biology of the cell. 21:2241-2256.
Nascimento, A. A., J. T. Roland, and V. I. Gelfand. 2003. Pigment cells: a model for the study of organelle transport. Annual review of cell and developmental biology. 19:469-491.
Nilsson, H., M. Rutberg, and M. Wallin. 1996. Localization of kinesin and cytoplasmic dynein in cultured melanophores from Atlantic cod, Gadus morhua. Cell motility and the cytoskeleton. 33:183-196.
Ohlin, M., I. Iranmanesh, A. E. Christakou, and M. Wiklund. 2015. Temperature-controlled MPapressure ultrasonic cell manipulation in a microfluidic chip. Lab on a chip. 15:3341-3349.
Orange, J. S., S. Roy-Ghanta, E. M. Mace, S. Maru, G. D. Rak, K. B. Sanborn, A. Fasth, R. Saltzman, A. Paisley, L. Monaco-Shawver, P. P. Banerjee, and R. Pandey. 2011. IL-2 induces a WAVE2-dependent pathway for actin reorganization that enables WASp-independent human NK cell function. The Journal of clinical investigation. 121:1535-1548.
Peruzzi, G., L. Femnou, A. Gil-Krzewska, F. Borrego, J. Weck, K. Krzewski, and J. E. Coligan. 2013. Membrane-type 6 matrix metalloproteinase regulates the activation-induced downmodulation of CD16 in human primary NK cells. Journal of immunology. 191:1883-1894.
Rak, G. D., E. M. Mace, P. P. Banerjee, T. Svitkina, and J. S. Orange. 2011. Natural killer cell lytic granule secretion occurs through a pervasive actin network at the immune synapse. PLoS biology. 9:e1001151.
Ramos, T. N., D. C. Bullard, and S. R. Barnum. 2014. ICAM-1: isoforms and phenotypes. Journal of immunology. 192:4469-4474.
Ren, Q., C. Wimmer, M. C. Chicka, S. Ye, Y. Ren, F. M. Hughson, and S. W. Whiteheart. 2010. Munc13-4 is a limiting factor in the pathway required for platelet granule release and hemostasis. Blood. 116:869-877.
Ritter, A. T., Y. Asano, J. C. Stinchcombe, N. M. Dieckmann, B. C. Chen, C. Gawden-Bone, S. van Engelenburg, W. Legant, L. Gao, M. W. Davidson, E. Betzig, J. Lippincott-Schwartz, and G. M. Griffiths. 2015. Actin depletion initiates events leading to granule secretion at the immunological synapse. Immunity. 42:864-876.
Stojanovic, A., and A. Cerwenka. 2011. Natural killer cells and solid tumors. Journal of innate immunity. 3:355-364.
Viswanath, D. I., E. M. Mace, H. T. Hsu, and J. S. Orange. 2016. Quantification of natural killer cell polarization and visualization of synaptic granule externalization by imaging flow cytometry. Clinical immunology.
Vivier, E., E. Tomasello, M. Baratin, T. Walzer, and S. Ugolini. 2008. Functions of natural killer cells. Nature immunology. 9:503-510.
Yi, J., X. Wu, A. H. Chung, J. K. Chen, T. M. Kapoor, and J. A. Hammer. 2013. Centrosome repositioning in T cells is biphasic and driven by microtubule end-on capture-shrinkage. The Journal of cell biology. 202:779-792.
Zhang, M., M. E. March, W. S. Lane, and E. O. Long. 2014. A signaling network stimulated by beta2 integrin promotes the polarization of lytic granules in cytotoxic cells. Science signaling. 7:ra96.
Zografou, S., D. Basagiannis, A. Papafotika, R. Shirakawa, H. Horiuchi, D. Auerbach, M. Fukuda, and S. Christoforidis. 2012. A complete Rab screening reveals novel insights in Weibel-Palade body exocytosis. Journal of cell science. 125:4780-4790.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of enhancing a cellular therapy for cancer for an individual, comprising the step of exposing cells of the cellular therapy to an effective amount of one or more agents that inhibits convergence of lytic granules in the cells, controls positioning of lytic granules in the cells, and/or maintains lytic granules near the surface of the cells.

2. The method of claim 1, wherein the cells are immune cells or cytotoxic cells.

3. The method of claim 2, wherein the immune cells are T cells, NK cells, NK T cells, cytotoxic innate lymphoid cells, or a mixture thereof.

4. The method of claim 1, wherein the cells are from cell lines.

5. The method of claim 1, wherein the cells are allogeneic or autologous to the individual.

6. The method of claim 1, wherein the one or more agents are exposed to the cells ex vivo.

7. The method of claim 1, wherein the one or more agents are expressed from a non-endogenous molecule in the cells.

8. The method of claim 7, wherein the non-endogenous molecule is an expression vector in the cell or a molecule that has incorporated into the genome of the cell.

9. The method of claim 1, wherein the one or more agents are one or more of the following:
a) an inhibitor of a motor protein involved in transport of the granules,
b) an inhibitor of an activating receptor of the motor protein;
c) an inhibitor of a signaling molecule for the motor protein;
d) an inhibitor of a receptor that induces a signaling molecule for the motor protein function;
e) an inhibitor of a molecule linking lytic granules to microtubules and/or motor proteins;
f) expression of a molecule in a cytotoxic cell that interferes with or eliminates dynein; and/or
g) an agent that eliminates the expression of a protein that facilitates granule convergence.

10. The method of claim 1, wherein the one or more agents are one or more of the following:
a) an inhibitor of dynein;
b) an inhibitor of an activating receptor of dynein;
c) an inhibitor of a signaling molecule for dynein function; and/or
d) an inhibitor of a receptor that induces a signaling molecule for dynein function.

11. The method of claim 1, wherein the inhibitor is an inhibitor of dynein, dynactin, HkRP3, Rab7, RILP, ORP1L, Pyk2, CLP170, leupaxin, LFA1, CD11a, CD18, CD54, Src, NIK, RASGRP1, PTEN, ILK, PINCH1, γ-parvin, paxillin, RhoGEF7; CDC42, Par6, aPKC, GSKβ, APC, IQGAP1, CLIP-170, Arl8b, or a combination thereof.

12. The method of claim 10, wherein the dynein that is inhibited is heavy chain, intermediate chain, light intermediate chain, or light chain.

13. The method of claim 10, wherein the dynein is DYNC1H1, DYNC2H1, DYNC1I1, DYNC1I2, DYNC1LI1, DYNC1LI2, DYNC2LI1, DYNLL1, DYNLL2, DYNLRB1, DYNLRB2, DYNLT1, or DYNLT3.

14. The method of claim 1, wherein the inhibitor of dynein is a ciliobrevin.

15. The method of claim 1, wherein cells of the cellular therapy exhibit a bystander effect on cells of the cancer.

16. A method of enhancing a therapy for cancer in an individual, comprising the step of administering to the individual an effective amount of one or more agents that inhibits convergence of lytic granules in the cells, controls positioning of lytic granules in the cells, or maintains lytic granules near the surface of the cells in immune cells or cytotoxic cells of the individual.

17. The method of claim 16, wherein the therapy is an antibody, a fragment of an antibody, a soluble ligand or receptor, a cell permeable peptide, a nucleic acid, a CRISPR/CASP9 construct, or a mixture thereof.

18. The method of claim 17, wherein the antibody is an anti-LFA-1 antibody, an anti-CD18 antibody, an antibody to CD11a, or a combination thereof.
